SEQUENCE LISTING (3 sequences)

SEQ ID NO: 1 - DNA, 19 bp, synthetic construct
  source 1..19: mol_type = other DNA
ctcgtggtggacttctctc

SEQ ID NO: 2 - DNA, 18 bp, synthetic construct
  source 1..18: mol_type = other DNA
ctgcaggatgaagaggaa

SEQ ID NO: 3 - DNA, 26 bp, synthetic construct
  source 1..26: mol_type = other DNA
  misc_feature <1: note = 5' 56-FAM
  misc_feature 9..10: note = internal quencher ZEN
  misc_feature >26: note = 3' quencher 3IABkFQ
tgtcctggttatcgctggatgtgtct

(19) United States
(12) Patent Application Publication
(10) Pub. No.: US 2025/0235462 A1
(43) Pub. Date: Jul. 24, 2025

(54) NOVEL CAPSID ASSEMBLY INHIBITORS

(71) Applicants: AM SCIENCES INC, Seoul (KR); KOREA RESEARCH INSTITUTE OF CHEMICAL TECHNOLOGY, Daejeon (KR)

(72) Inventors: Nakcheol JEONG, Seoul (KR); Misun LEE, Seoul (KR); Heewoo SIM, Seoul (KR); Myungjin KIM, Seoul (KR); Jung-Hee KIM, Seoul (KR); Soo Bong HAN, Daejeon (KR); Young Sik JUNG, Daejeon (KR); Hye jin KIM, Daejeon (KR); Chang Soo YUN, Daejeon (KR); Heeyeong CHO, Daejeon (KR); Mee Hyein KIM, Daejeon (KR); Joo Youn LEE, Daejeon (KR)

(21) Appl. No.: 18/699,182

(22) PCT Filed: Oct. 12, 2022
(86) PCT No.: PCT/KR2022/015335

§ 371 Date: Apr. 5, 2024

(30) Foreign Application Priority Data
Oct. 12, 2021 (KR) 10-2021-0134940

(51) Int. Cl. A61K 31/55; A61K 31/437; C07D 487/04

(52) U.S. Cl. CPC A61K 31/55; A61K 31/437; C07D 487/04

(57) ABSTRACT

The present invention relates to a series of novel triazolomethylurea derivatives, and the use thereof for the inhibition of capsid assembly and the prevention or treatment of viral diseases through the inhibition.

### TECHNICAL FIELD

The present invention relates to a series of novel triazolomethylurea derivatives, and the use thereof for the inhibition of capsid assembly and the prevention or treatment of viral diseases through the inhibition.

### BACKGROUND ART

Chronic hepatitis B virus (HBV) infection is a major health problem worldwide and may cause serious health problems such as cirrhosis or liver cancer. According to a recent WHO report, it is estimated that 257 million people worldwide are living with chronic HBV infection, and it is estimated that 1.34 million die each year due to hepatitis-related complications. To date, substances approved for the treatment of HBV include interferons (IFNs, non-pegylated or pegylated) and nucleos(t)ide analogues, lamivudine, adefovir, entecavir, tenofovir and the like. IFN therapy induces suppression of HBV replication and remission of liver disease, and nucleos(t)ide drugs suppress reverse transcriptase and DNA polymerase activity. Nucleoside analogues effectively control viral proliferation, but the viral gene, which is the key to HBV, remains together in the form of cccDNA as in the form of the host's mini-chromosome, and thus its complete elimination is difficult. Therefore, drug resistance occurs frequently in HBV carriers because they should use drugs for a long time to prevent the growth of new virus. In order to overcome this unmet medical need, there is a need for the discovery of efficient and safe anti-HBV drugs with novel molecular targets.

The HBV core proteins play an important role in the viral life cycle. The HBV capsid formed by the assembly of core proteins encapsulates pregenomic RNA (pgRNA), reverse transcriptase, and DNA polymerase together to modulate reverse transcription of pgRNA and recycling of nucleocapsids. The core proteins modulate the transport and nuclear release of viral genome, are involved in epigenetic regulation of cccDNA to modulate host gene expression. Hence, based on the HBV replication cycle, the modulation of the action of core proteins and the discovery of target anti-HBV agents, which prevent the formation of capsid proteins composed of core proteins, have been extensively studied.

A number of research centers and pharmaceutical companies have developed capsid assembly modulators. Bay-41-4109, a heteroaryldihydropyrimidine (HAP) analog, is the first capsid assembly inhibitor to enter clinical trials, and induces aberrant formation of capsids and aggregation of capsid proteins. As a result of incorrect assembly of capsid proteins, it was observed in HepG2.2.15 cells that HBV core protein was degraded by proteasome (proteasome mediated degradation) together with HBV DNA reduction by inhibiting HBV DNA replication. GLS-4 is a second-generation HAP analog having the same mechanism of action as BAY-41-4109, and is in progress in phase II clinical trials together with ritonavir (RTV) in order to prevent the induction of CYP enzymes by GLS-4. The same researchers have reported HEC72702 with reduced CYP enzyme induction, lower suppression of hERG K⁺ channels, and improved oral bioavailability while sacrificing some in vitro potency.

Other types of capsid assembly modulators, AT130, NVR 3-778, and JNJ-632, with a different mechanism of action from that of HAPs, have also been reported. For example, when capsid is treated with NVR 3-778, capsid is normally formed, but an empty capsid into which pregenomic RNA (pgRNA), reverse transcriptase, and DNA polymerase are not included is obtained. JNJ-632 has been reported as a novel sulfamoylbenzamide capsid assembly modulator, and its optimized analog JNJ-6379 is currently in phase II clinical trials. Recently, GIST researchers Kang, J. A. et al. have reported ciclopirox, an FDA-approved antifungal drug, as an orally available capsid assembly inhibitor in a drug repositioning strategy.

### CITATION LIST

### Patent Documents

WO 2017/210545; and

US 2017/0355708

### Non-Patent Document

Kang, J. A. et al., Nat. Commun., 2019, 10(2184):1-14

### DISCLOSURE OF INVENTION

### Technical Problem

As a result of intensive research efforts to discover novel small molecule compounds that can suppress viral infection by inhibiting capsid assembly, the present inventors have confirmed that a series of triazolomethylurea derivatives have the activity of suppressing viral infection by potentially inhibiting capsid assembly, thus completing the present invention.

### Solution to Problem

An object of the present invention is to provide a compound represented by the following Chemical Formula 1 or a pharmaceutically acceptable salt of the compound:

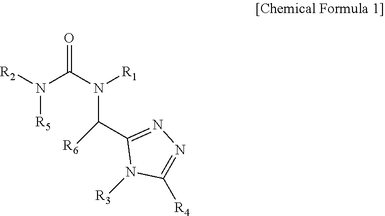

in Chemical Formula 1,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₅ and R₆ are each independently hydrogen or C₁₋₄ alkyl; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkoxy-carbonyl-C₁₋₄ alkyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, C₆₋₁₀ aryl, C₃₋₁₀ cycloalkyl, C₆₋₁₀ aryl-C₁₋₄ alkoxycarbonyl, and 5- to 10-membered heteroaryl.

Another object of the present invention is to provide a method for preparing the compound described above or a pharmaceutically acceptable salt thereof, which includes reacting a compound represented by the following Chemical Formula 2 with a compound represented by the following Chemical Formula 3:

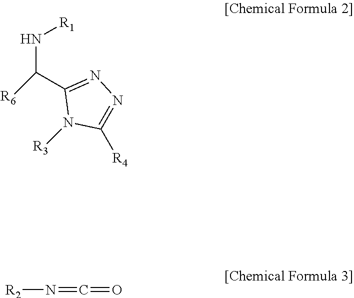

in Chemical Formulas 2 and 3,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₆ is hydrogen or C₁₋₄ alkyl; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkoxy-carbonyl-C₁₋₄ alkyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, C₆₋₁₀ aryl, C₃₋₁₀ cycloalkyl, C₆₋₁₀ aryl-C₁₋₄ alkoxycarbonyl, and 5- to 10-membered heteroaryl.

Still another object of the present invention is to provide a composition for capsid assembly inhibition, containing the compound described above or a pharmaceutically acceptable salt thereof.

Still another object of the present invention is to provide an antiviral composition containing the compound described above or a pharmaceutically acceptable salt thereof as an active ingredient.

Still another object of the present invention is to provide a pharmaceutical composition for prevention or treatment of viral disease, containing the compound described above or a pharmaceutically acceptable salt thereof as an active ingredient.

Still another object of the present invention is to provide a method for treating a viral disease, which includes administering the pharmaceutical composition described above to an individual in need thereof.

### Advantageous Effects of Invention

The triazolomethylurea derivatives in the molecule newly synthesized according to the present invention exhibit low cytotoxicity and an effect of inhibiting capsid assembly, and can be thus usefully used to prevent or treat diseases related to capsid assembly, for example, viral diseases caused by HBV, HCV, HIV, and the like.

### BEST MODE FOR CARRYING OUT THE INVENTION

Each description and embodiment disclosed in this disclosure may also be applied to other descriptions and embodiments. That is, all combinations of various elements disclosed in this disclosure fall within the scope of the present disclosure. Further, the scope of the present disclosure is not limited by the specific description below.

Further, those skilled in the art will recognize, or be able to ascertain using no more than routine experimentation, many equivalents to the specific embodiments of the invention described herein. Further, these equivalents should be interpreted to fall within the scope of the present invention.

In addition, throughout this specification, when a part is referred to as “including” an element, it will be understood that other elements may be further included rather than other elements being excluded unless content to the contrary is specially described.

Hereinafter, the present invention will be described in detail.

A first aspect of the present invention provides a compound represented by the following Chemical Formula 1 or a pharmaceutically acceptable salt of the compound:

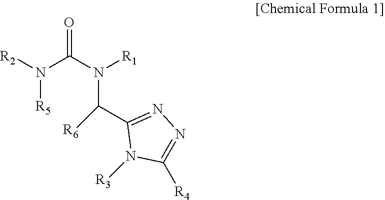

in Chemical Formula 1,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₅ and R₆ are each independently hydrogen or C₁₋₄ alkyl; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkoxy-carbonyl-C₁₋₄ alkyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, C₆₋₁₀ aryl, C₃₋₁₀ cycloalkyl, C₆₋₁₀ aryl-C₁₋₄ alkoxycarbonyl, and 5- to 10-membered heteroaryl.

For example, in Chemical Formula 1, R₁ may be phenyl, benzodioxolyl, dihydropyridinyl, cyclopentyl, indolyl, benzothiazolyl, pyrazolyl, isoxazolyl, oxazolyl, or pyridinyl, but is not limited thereto.

For example, in Chemical Formula 1, R₂ may be phenyl, cyclopentyl, or pyridinyl, but is not limited thereto.

For example, in Chemical Formula 1, R₃ may be hydrogen, methyl, phenyl, isopropyl, cyclopropyl, cyclopentyl, hydroxyethyl, or methoxyethyl, but is not limited thereto.

For example, in Chemical Formula 1, R₄ may be methyl, isopropyl, phenyl, cyclopentyl, methoxycarbonylmethyl, difluoromethyl, or trifluoromethyl, but is not limited thereto.

For example, in Chemical Formula 1, R₃ and R₄ may be connected to each other to form azepanyl, morpholinyl, oxazepanyl, diazepanyl, or piperidinyl including carbon and nitrogen to which R₃ and R₄ are bonded, but are not limited thereto.

For example, in Chemical Formula 1, R₅ may be hydrogen or methyl, but is not limited thereto.

For example, in Chemical Formula 1, R₆ may be hydrogen or methyl, but is not limited thereto.

For example, in Chemical Formula 1, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other may be unsubstituted or substituted with one or more selected from the group consisting of cyano, hydroxy, fluoro, chloro, methyl, isopropyl, methoxy, isopropoxy, methoxyethyl, tert-butoxycarbonyl, methoxycarbonylmethyl, hydroxyethyl, difluoromethyl, trifluoromethyl, methylamino, dimethylamino, cyclopropyl, cyclopentyl, benzyloxycarbonyl, and phenyl, but is not limited thereto.

Specifically, the compound may be
1. 1-(benzo[d][1,3]dioxol-5-yl)-1-((6,7,8,9-tetrahydro-5H-[1,2,4]triazolo[4,3-a]azepin-3-yl)methyl)-3-m-tolylurea,
2. 1-(benzo[d][1,3]dioxol-5-yl)-3-(3-chloro-4-fluorophenyl)-1-((6,7,8,9-tetrahydro-5H-[1,2,4]triazolo[4,3-a]azepin-3-yl)methyl)urea,
3. 3-(3-chloro-4-fluorophenyl)-1-(4-methoxyphenyl)-1-((6,7,8,9-tetrahydro-5H-[1,2,4]triazolo[4,3-a]azepin-3-yl)methyl)urea,
4. 1-(benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea,
5. 1-(benzo[d][1,3]dioxol-5-yl)-3-(2-chlorophenyl)-1-((6,7,8,9-tetrahydro-5H-[1,2,4]triazolo[4,3-a]azepin-3-yl)methyl)urea,
6. 3-(3-chloro-4-fluorophenyl)-1-cyclopentyl-1-((6,7,8,9-tetrahydro-5H-[1,2,4]triazolo[4,3-a]azepin-3-yl)methyl)urea,
7. 1-(benzo[d][1,3]dioxol-5-yl)-3-(3-chloro-4-fluorophenyl)-1-((5,6,8,9-tetrahydro-[1,2,4]triazolo[4,3-d][1,4]oxazepin-3-yl)methyl)urea,
8. 1-(benzo[d][1,3]dioxol-5-yl)-3-(3-cyanophenyl)-1-((6,7,8,9-tetrahydro-5H-[1,2,4]triazolo[4,3-a]azepin-3-yl)methyl)urea,
9. 1-(3-chloro-4-fluorophenyl)-3-(4-methoxyphenyl)-1-methyl-3-((6,7,8,9-tetrahydro-5H-[1,2,4]triazolo[4,3-a]azepin-3-yl)methyl)urea,
10. 3-(3-chloro-4-fluorophenyl)-1-(1H-indol-6-yl)-1-((6,7,8,9-tetrahydro-5H-[1,2,4]triazolo[4,3-a]azepin-3-yl)methyl)urea,
11. 1-(benzo[d][1,3]dioxol-5-yl)-3-cyclopentyl-1-((6,7,8,9-tetrahydro-5H-[1,2,4]triazolo[4,3-a]azepin-3-yl)methyl)urea,
12. 3-(3-chloro-4-fluorophenyl)-1-(4-isopropoxyphenyl)-1-((6,7,8,9-tetrahydro-5H-[1,2,4]triazolo[4,3-a]azepin-3-yl)methyl)urea,
13. 3-(3-chloro-4-fluorophenyl)-1-((6,7,8,9-tetrahydro-5H-[1,2,4]triazolo[4,3-a]azepin-3-yl)methyl)-1-(4-(trifluoromethyl)phenyl)urea,
14. 3-(3-chloro-4-fluorophenyl)-1-(4-(methylamino)phenyl)-1-((6,7,8,9-tetrahydro-5H-[1,2,4]triazolo[4,3-a]azepin-3-yl)methyl)urea,
15. 3-(3-chloro-4-fluorophenyl)-1-((4,5-dimethyl-4H-1,2,4-triazol-3-yl)methyl)-1-(4-methoxyphenyl)urea,
16. 1-(benzo[d]thiazol-6-yl)-3-(3-chloro-4-fluorophenyl)-1-((6,7,8,9-tetrahydro-5H-[1,2,4]triazolo[4,3-a]azepin-3-yl)methyl)urea,
17. tert-butyl 5-(3-(3-chloro-4-fluorophenyl)-1-((6,7,8,9-tetrahydro-5H-[1,2,4]triazolo[4,3-a]azepin-3-yl)methyl)ureido)-1H-indole-1-carboxylate,
18. 3-(3-chloro-4-fluorophenyl)-1-(1H-indol-5-yl)-1-((6,7,8,9-tetrahydro-5H-[1,2,4]triazolo[4,3-a]azepin-3-yl)methyl)urea,
19. 3-(3-chloro-4-fluorophenyl)-1-(3-cyanophenyl)-1-((6,7,8,9-tetrahydro-5H-[1,2,4]triazolo[4,3-a]azepin-3-yl)methyl)urea,
20. 1-(benzo[d][1,3]dioxol-5-yl)-3-(6-methylpyridin-2-yl)-1-((6,7,8,9-tetrahydro-5H-[1,2,4]triazolo[4,3-a]azepin-3-yl)methyl)urea,
21. 3-(3-chloro-4-fluorophenyl)-1-((6,7,8,9-tetrahydro-5H-[1,2,4]triazolo[4,3-a]azepin-3-yl)methyl)-1-(3-(trifluoromethyl)phenyl)urea,
22. 3-(3-chloro-4-fluorophenyl)-1-(3,4-difluorophenyl)-1-((6,7,8,9-tetrahydro-5H-[1,2,4]triazolo[4,3-a]azepin-3-yl)methyl)urea,
23. 3-(3-chloro-4-fluorophenyl)-1-(4-methoxyphenyl)-1-(1-(6,7,8,9-tetrahydro-5H-[1,2,4]triazolo[4,3-a]azepin-3-yl)ethyl)urea,
24. 3-(3-chloro-4-fluorophenyl)-1-(4-methoxyphenyl)-1-((4-methyl-5-phenyl-4H-1,2,4-triazol-3-yl)methyl)urea,
25. 3-(3-chloro-4-fluorophenyl)-1-(4-methoxyphenyl)-1-((5-methyl-4-phenyl-4H-1,2,4-triazol-3-yl)methyl)urea,
26. 3-(3-chloro-4-fluorophenyl)-1-((4-isopropyl-5-methyl-4H-1,2,4-triazol-3-yl)methyl)-1-(4-methoxyphenyl)urea,
27. 3-(3-chloro-4-fluorophenyl)-1-(4-methoxyphenyl)-1-((5,6,8,9-tetrahydro-[1,2,4]triazolo[4,3-d][1,4]oxazepin-3-yl)methyl)urea,
28. 3-(3-chloro-4-fluorophenyl)-1-(4-cyanophenyl)-1-((6,7,8,9-tetrahydro-5H-[1,2,4]triazolo[4,3-a]azepin-3-yl)methyl)urea,
29. 3-(3-chloro-4-fluorophenyl)-1-((5-isopropyl-4-methyl-4H-1,2,4-triazol-3-yl)methyl)-1-(4-methoxyphenyl)urea,
30. 1-(benzo[d][1,3]dioxol-5-yl)-3-(3-chloro-4-fluorophenyl)-1-((5,6,7,8,9,10-hexahydro-[1,2,4]triazolo[4,3-a]azocin-3-yl)methyl)urea,
31. 3-(3-chloro-4-fluorophenyl)-1-((5-cyclopentyl-4-methyl-4H-1,2,4-triazol-3-yl)methyl)-1-(4-methoxyphenyl)urea,
32. 3-(3-chloro-4-fluorophenyl)-1-((4-cyclopentyl-5-methyl-4H-1,2,4-triazol-3-yl)methyl)-1-(4-methoxyphenyl)urea,
33. 1-(benzo[d][1,3]dioxol-5-yl)-3-(3-chloro-4-fluorophenyl)-1-(1-(5,6,8,9-tetrahydro-[1,2,4]triazolo[4,3-d][1,4]oxazepin-3-yl)ethyl)urea,
34. 3-(3-chloro-4-fluorophenyl)-1-(4-(dimethylamino)phenyl)-1-((6,7,8,9-tetrahydro-5H-[1,2,4]triazolo[4,3-a]azepin-3-yl)methyl)urea,
35. 3-(3-chloro-4-fluorophenyl)-1-(1-methyl-1H-pyrazol-3-yl)-1-((6,7,8,9-tetrahydro-5H-[1,2,4]triazolo[4,3-a]azepin-3-yl)methyl)urea,
36. 3-(3-chloro-4-fluorophenyl)-1-(isoxazol-3-yl)-1-((6,7,8,9-tetrahydro-5H-[1,2,4]triazolo[4,3-a]azepin-3-yl)methyl)urea,
37. 3-(3-chloro-4-fluorophenyl)-1-((4-cyclopropyl-5-methyl-4H-1,2,4-triazol-3-yl)methyl)-1-(4-methoxyphenyl)urea,
38. benzyl 3-((3-(3-chloro-4-fluorophenyl)-1-(4-methoxyphenyl)ureido)methyl)-8,9-dihydro-5H-[1,2,4]triazolo[4,3-d][1,4]diazepine-7(6H)-carboxylate,
39. 3-(3-chloro-4-fluorophenyl)-1-(oxazol-2-yl)-1-((6,7,8,9-tetrahydro-5H-[1,2,4]triazolo[4,3-a]azepin-3-yl)methyl)urea,
40. 3-(3-chloro-4-fluorophenyl)-1-(6-methoxypyridin-3-yl)-1-((5,6,8,9-tetrahydro-[1,2,4]triazolo[4,3-d][1,4]oxazepin-3-yl)methyl)urea,
41. 1-(benzo[d][1,3]dioxol-5-yl)-3-(3-chlorophenyl)-1-((5,6,7,8-tetrahydro-[1,2,4]triazolo[4,3-a]pyridin-3-yl)methyl)urea,
42. 1-(benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea,
43. 3-(3-chloro-4-fluorophenyl)-1-(4-methoxyphenyl)-1-((5,6,7,8-tetrahydro-[1,2,4]triazolo[4,3-a]pyridin-3-yl)methyl)urea,
44. 3-(3-chloro-4-fluorophenyl)-1-(6-methoxypyridin-3-yl)-1-((6,7,8,9-tetrahydro-5H-[1,2,4]triazolo[4,3-a]azepin-3-yl)methyl)urea,
45. methyl 2-(5-((1-(benzo[d][1,3]dioxol-5-yl)-3-(3-chloro-4-fluorophenyl)ureido)methyl)-4H-1,2,4-triazol-3-yl)acetate,
46. 3-(3-chloro-4-fluorophenyl)-1-(2-hydroxypyridin-4-yl)-1-((5,6,8,9-tetrahydro-[1,2,4]triazolo[4,3-d][1,4]oxazepin-3-yl)methyl)urea,
47. 3-(3-chloro-4-fluorophenyl)-1-(2-methoxypyridin-4-yl)-1-((5,6,8,9-tetrahydro-[1,2,4]triazolo[4,3-d][1,4]oxazepin-3-yl)methyl)urea,
48. 3-(3-chloro-4-fluorophenyl)-1-((5-(difluoromethyl)-4-methyl-4H-1,2,4-triazol-3-yl)methyl)-1-(6-methoxypyridin-3-yl)urea,
49. 3-(3-chloro-4-fluorophenyl)-1-(6-methoxypyridin-3-yl)-1-((4-methyl-5-(trifluoromethyl)-4H-1,2,4-triazol-3-yl)methyl)urea,
50. 3-(3-chloro-4-fluorophenyl)-1-((4-(2-hydroxyethyl)-5-(trifluoromethyl)-4H-1,2,4-triazol-3-yl)methyl)-1-(6-methoxypyridin-3-yl)urea,
51. 3-(3-chloro-4-fluorophenyl)-1-((5-(difluoromethyl)-4-(2-methoxyethyl)-4H-1,2,4-triazol-3-yl)methyl)-1-(6-methoxypyridin-3-yl)urea,
52. 3-(3-chloro-4-fluorophenyl)-1-((4-(2-methoxyethyl)-5-(trifluoromethyl)-4H-1,2,4-triazol-3-yl)methyl)-1-(6-methoxypyridin-3-yl)urea, or
53. 3-(3-chloro-4-fluorophenyl)-1-((5-(difluoromethyl)-4-(2-hydroxyethyl)-4H-1,2,4-triazol-3-yl)methyl)-1-(6-methoxypyridin-3-yl)urea, but is not limited thereto.

The compounds of the present invention may exist in the form of pharmaceutically acceptable salts. As the salts, acid addition salts formed by pharmaceutically acceptable free acids are useful. As used herein, the term “pharmaceutically acceptable salt” means all organic or inorganic addition salts of the compounds which have a concentration that is relatively non-toxic and harmless to patients and exhibits an effective action, and which cause side effects that do not diminish the beneficial efficacy of the compound represented by Chemical Formula 1.

An acid addition salt is prepared by way of a conventional method, for example, by dissolving the compound in an excess amount of an aqueous acid solution and precipitating this salt using a water-miscible organic solvent such as methanol, ethanol, acetone, or acetonitrile. Equimolar amounts of the compound and an acid or alcohol in water (for example, glycol monomethyl ether) may be heated, and then the mixture may be evaporated to dryness, or the precipitated salt may be subjected to suction filtration.

Here, organic acids and inorganic acids may be used as free acids. hydrochloric acid, phosphoric acid, sulfuric acid, nitric acid, tartaric acid and the like may be used as the inorganic acids. Methanesulfonic acid, p-toluenesulfonic acid, acetic acid, trifluoroacetic acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like may be used as the organic acids. The free acids are not limited thereto.

The pharmaceutically acceptable metal salts may be prepared using bases. Alkali metal salts or alkaline earth metal salts are obtained by, for example, dissolving the compound in an excess amount of an alkali metal hydroxide or alkaline earth metal hydroxide solution, filtering the undissolved compound salt, and then evaporating and drying the filtrate. Here, it is pharmaceutically suitable to prepare a sodium, potassium, or calcium salt as the metal salts, but the metal salts are not limited thereto. A corre-sponding silver salt may be obtained by reacting an alkali metal or alkaline earth metal salt with a suitable silver salt (for example, silver nitrate).

The pharmaceutically acceptable salts of the compound of the present invention include salts of acidic or basic groups that may be present in the compound represented by Chemical Formula 1 unless otherwise indicated. For example, the pharmaceutically acceptable salts may include sodium, calcium, and potassium salts of hydroxyl group, and the like, other pharmaceutically acceptable salts of amino group may include hydrobromide, sulfate, hydrogen sulfate, phosphate, hydrogen phosphate, dihydrogen phosphate, acetate, succinate, citrate, tartrate, lactate, mandelate, methanesulfonate (mesylate), and p-toluenesulfonate (tosylate) salts, and these salts may be prepared by way of methods for preparing salts known in the art.

As the salts of the triazolomethylurea derivative compounds of the present invention, all salts of triazolomethylurea derivative compounds that are pharmaceutically acceptable salts and exhibit pharmacological activity equivalent to that of the triazolomethylurea derivative compounds may be used without limitation.

The compound represented by Chemical Formula 1 according to the present invention includes not only pharmaceutically acceptable salts thereof but also solvates such as hydrates that can be prepared from the salts and all possible stereoisomers without limitation. The solvates and stereoisomers of the compound represented by Chemical Formula 1 may be prepared from the compound represented by Chemical Formula 1 by methods known in the art.

Furthermore, the compound represented by Chemical Formula 1 according to the present invention may be prepared in a crystalline form or an amorphous form, and may optionally be hydrated or solvated when prepared in a crystalline form. In the present invention, compounds containing various amounts of water as well as stoichiometric hydrates of the compound represented by Chemical Formula 1 may be included. The solvates of the compound represented by Chemical Formula 1 according to the present invention include both stoichiometric solvates and non-stoichiometric solvates.

A second aspect of the present invention provides a method for preparing the compound of the first aspect or a pharmaceutically acceptable salt thereof, which includes reacting a compound represented by the following Chemical Formula 2 with a compound represented by the following Chemical Formula 3:

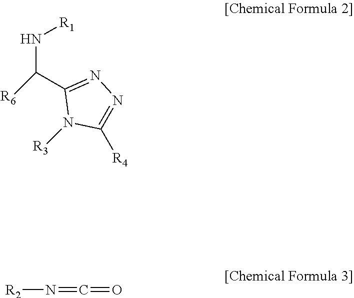

in Chemical Formulas 2 and 3,
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₆ is hydrogen or C₁₋₄ alkyl; and
here, a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkoxy-carbonyl-C₁₋₄ alkyl, C₁₋₄ alkylaminosulfonyl, C₁₋₄ alkylsulfonylamino, C₁₋₄ hy-droxyalkyl, C₁₋₄ haloalkyl, C₁₋₄ alkylamino, di(C₁₋₄ alkyl)amino, C₆₋₁₀ aryl, C₃₋₁₀ cycloalkyl, C₆₋₁₀ aryl-C₁₋₄ alkoxycarbonyl, and 5- to 10-membered heteroaryl.

For example, the preparation method of the present invention may be performed in an organic solvent in the presence of 4-dimethylaminopyridine (DMAP), but is not limited thereto.

For example, the compound represented by Chemical Formula 2 may be prepared by reacting i) a compound represented by the following Chemical Formula 4 with a compound represented by Chemical Formula 5 or ii) a compound represented by the following Chemical Formula 6 with a compound represented by Chemical Formula 7, but is not limited thereto:

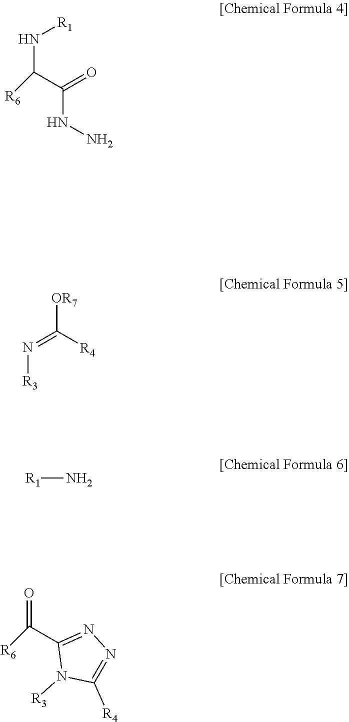

in Chemical Formula 5,
R₇ is C₁₋₄ alkyl.

For example, in the preparation method of the present invention, a step of conducting a reaction with R₅—X (R₅ is hydrogen or C₁₋₄ alkyl and X is halogen) may be further performed after the reaction of the compound represented by Chemical Formula 2 with the compound represented by Chemical Formula 3 in order to introduce a substituent.

A third aspect of the present invention provides a composition for capsid assembly inhibition, containing the compound of the first aspect or a pharmaceutically acceptable salt thereof.

As used herein, the terms “compound of the first aspect” and “pharmaceutically acceptable salt” are as described above.

As used herein, the term “capsid” refers to a protein construct of a virus surrounding the genetic materials and enzymes required for reverse transcription, and is composed of subunits of several oligomeric (repetitive) structures of proteins called protomers. Observable three-dimensional morphological subunits that may or may not correspond to individual proteins are called capsomeres. The proteins that constitute the capsid are called capsid proteins or viral coat proteins (VCP). The capsid and its genome are called nucleocapsids. The capsids are broadly classified depending on their structures, and most viruses have capsids of a helical or icosahedral structure. Some viruses, such as bacteriophages, have developed into more complex structures because of the limitations in elasticity and electrostatics. The capsid surface may be composed of one or more proteins; for example, a foot-and-mouth disease virus capsid has a surface composed of the three proteins VP1-3. When a virus infects a cell and begins replicating itself, a new capsid subunit is synthesized using the protein biosynthesis mechanism of the cell. The genetic material encapsulated by the capsid may be RNA or DNA, but is not limited thereto.

For example, when infected with a virus, the host cell must rapidly produce thousands of identical copies of the original virus. When not inside an infected cell or in the process of infecting a cell, the virus exists in the form of (i) a genetic material, namely, long molecules of DNA or RNA that encode the protein needed by the virus to proliferate itself; (ii) capsids that are protein coats to surround and protect the genetic material; and optionally (iii) independent particles or virions that are surrounded by a lipid envelope and define viral identity.

A fourth aspect of the present invention provides an antiviral composition containing the compound of the first aspect or a pharmaceutically acceptable salt thereof as an active ingredient.

As used herein, the terms “compound of the first aspect” and “pharmaceutically acceptable salt” are as described above.

A fifth aspect of the present invention provides a pharmaceutical composition for prevention or treatment of viral disease, containing the compound of the first aspect or a pharmaceutically acceptable salt thereof as an active ingredient.

As used herein, the terms “compound of the first aspect” and “pharmaceutically acceptable salt” are as described above.

As used herein, the term “prevention” means any action in which the occurrence, spread, and recurrence of a viral disease is suppressed or delayed by administration of the composition of the present invention, and the term “treatment” means any action in which the symptoms of the disease are improved or advantageously changed by administration of the composition of the present invention.

The pharmaceutical composition of the present invention can prevent or treat diseases caused by viral infection by promoting the formation of an abnormal capsid in which the genetic material and elements replicating the genetic material are removed.

The viral disease may be an infectious disease caused by hepatitis B virus (HBV), hepatitis C virus (HCV), or human immunodeficiency virus (HIV), but is not limited thereto.

The pharmaceutical composition according to the present invention may contain the compound represented by Chemical Formula 1 or a pharmaceutically acceptable salt thereof as an active ingredient at preferably 0.1% to 75% by weight, more preferably 1% to 50% by weight based on the total weight of the composition.

The composition of the present invention may further contain a pharmaceutically acceptable carrier, diluent, or excipient, may be formulated and used in various forms such as oral formulations such as powders, granules, tablets, capsules, suspensions, emulsions, syrups, and aerosols and injections of sterile injection solutions by way of conventional methods depending on each purpose of use, and may be administered orally or through various routes including intravenous, intraperitoneal, subcutaneous, rectal, topical, and the like. Examples of the suitable carrier, excipient, or diluent that may be contained in such a composition include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oil. The composition of the present invention may further contain a filler, an anti-agglomeration agent, a lubricant, a wetting agent, a flavoring agent, an emulsifier, a preservative, and the like.

Solid preparations for oral administration include tablets, pills, powders, granules, capsules, and the like, and such solid preparations are formulated by mixing at least one or more excipients such as starch, calcium carbonate, sucrose, lactose, or gelatin with the composition. In addition to simple excipients, lubricants such as magnesium stearate and talc may be used.

Liquid preparations for oral administration may include suspensions, internal solutions, emulsions, and syrups, and may contain various excipients such as wetting agents, sweetening agents, fragrances, and preservatives in addition to water and liquid paraffin that are commonly used simple diluents.

Preparations for parenteral administration include sterilized aqueous solutions, non-aqueous solutions, suspensions, emulsions, freeze-dried preparations, and suppositories. As non-aqueous solvents and suspending agents, propylene glycol, polyethylene glycol, vegetable oils such as olive oil, injectable esters such as ethyl oleate, and the like may be used. As the base of suppositories, Witepsol, Macrogol, Tween 61, cacao butter, laurin, glycerogelatin and the like may be used. Meanwhile, the injections may contain conventional additives such as solubilizers, isotonic agents, suspending agents, emulsifiers, stabilizers, and preservatives.

Here, the composition of the present invention is administered in a pharmaceutically effective amount. As used herein, the term “pharmaceutically effective amount” refers to an amount sufficient to treat a disease at a reasonable benefit/risk ratio applicable to medical treatment and not to cause side effects, and the effective dose level may be determined depending on factors including patient's health condition, the kind and severity of disease, drug activity, sensitivity to drug, method of administration, time of administration, route of administration and excretion rate, duration of treatment, mixing, or concomitant drugs, and other factors well known in the medical art. The composition of the present invention may be administered as an individual therapeutic agent or in combination with other therapeutic agents, may be administered sequentially or simultaneously with conventional therapeutic agents, and may be administered in a single or multiple manner. It is important to administer the composition in an amount so that the maximum effect can be acquired with a minimum amount without side effects in consideration of all the above factors, and the amount can be easily determined by those skilled in the art.

For example, the pharmaceutically effective amount may be increased or decreased depending on the route of administration, the severity of disease, gender, weight, age, and the like, and thus the dosage is not intended to limit the scope of the present invention in any way.

Specifically, the effective amount of the compound in the composition of the present invention may vary depending on the age, gender, and weight of the patient, and the compound may be administered generally at 1 mg to 100 mg, preferably 5 mg to 60 mg per kg of weight every day or every other day or 1 to 3 times a day in a divided manner. However, the effective amount may be increased or decreased depending on the route of administration, the severity of disease, gender, weight, age, and the like, and thus the dosage is not intended to limit the scope of the present invention in any way.

A sixth aspect of the present invention provides a method for treating a viral disease, which includes administering the pharmaceutical composition of the fifth aspect to an individual in need thereof.

As used herein, the terms “pharmaceutical composition of the fifth aspect” and “viral disease” are the same as described above.

As used herein, the term “individual” refers to all animals including monkeys, cows, horses, sheep, pigs, chickens, turkeys, quails, cats, dogs, mice, rats, rabbits, or guinea pigs, and humans, who have or may develop the viral diseases. By administering the pharmaceutical composition of the present invention to an individual, the disease can be effectively prevented or treated. The pharmaceutical composition of the present invention may be administered in combination with a conventional therapeutic agent.

As used herein, the term “administration” means to supply a predetermined substance to a patient by way of an arbitrary suitable method, and the composition of the present invention may be administered through any general route as long as it can reach the target tissue. The administration may be intraperitoneal administration, intravenous administration, intramuscular administration, subcutaneous administration, intradermal administration, oral administration, topical administration, intranasal administration, intrapulmonary administration, or rectal administration, but is not limited thereto. The pharmaceutical composition of the present invention may be administered using an arbitrary device capable of transporting an active substance to a target cell. Preferred administration modes and preparations are intravenous injections, subcutaneous injections, intradermal injections, intramuscular injections, drip injections, and the like. The injections may be prepared using aqueous solvents such as physiological saline solution and Ringer's solution, non-aqueous solvents such as vegetable oil, higher fatty acid esters (for example, ethyl oleate), alcohols (for example, ethanol, benzyl alcohol, propylene glycol, and glycerin), and the like. The injections may contain pharmaceutical carriers such as stabilizers to prevent deterioration (for example, ascorbic acid, sodium hydrogen sulfite, sodium pyrosulfite, BHA, tocopherol, and EDTA), emulsifiers, buffers for pH adjustment, and preservatives to inhibit the growth of microorganisms (for example, phenylmercuric nitrate, thimerosal, benzalkonium chloride, phenol, cresol, and benzyl alcohol).

As used herein, the term “therapeutically effective amount” used in combination with an active ingredient means an amount of a triazolomethylurea derivative compound or a pharmaceutically acceptable salt thereof effective in the prevention or treatment of a target disease.

The pharmaceutical composition of the present invention may further contain a known drug used for the prevention or treatment of each known disease other than the triazolomethylurea derivative compound or a pharmaceutically acceptable salt thereof as an active ingredient depending on the kind of disease to be prevented or treated. For example, when used for the prevention or treatment of viral diseases, the pharmaceutical composition of the present invention may further contain a known drug other than the triazolomethylurea derivative compound or a pharmaceutically acceptable salt thereof as an active ingredient, and may be used in combination with other known treatments for the treatment of these diseases.

### MODE FOR THE INVENTION

Hereinafter, the configuration and effects of the present invention will be described in more detail with reference to exemplary embodiments. However, these exemplary embodiments are for illustrative purposes only, and the scope of the present invention is not intended to be limited by these exemplary embodiments.

### Example 1. 1-(Benzo[d][1,3]dioxol-5-yl)-1-((6,7,8,9-tetrahydro-5H-[1,2,4]triazolo[4,3-a]azepin-3-yl)methyl)-3-m-tolylurea

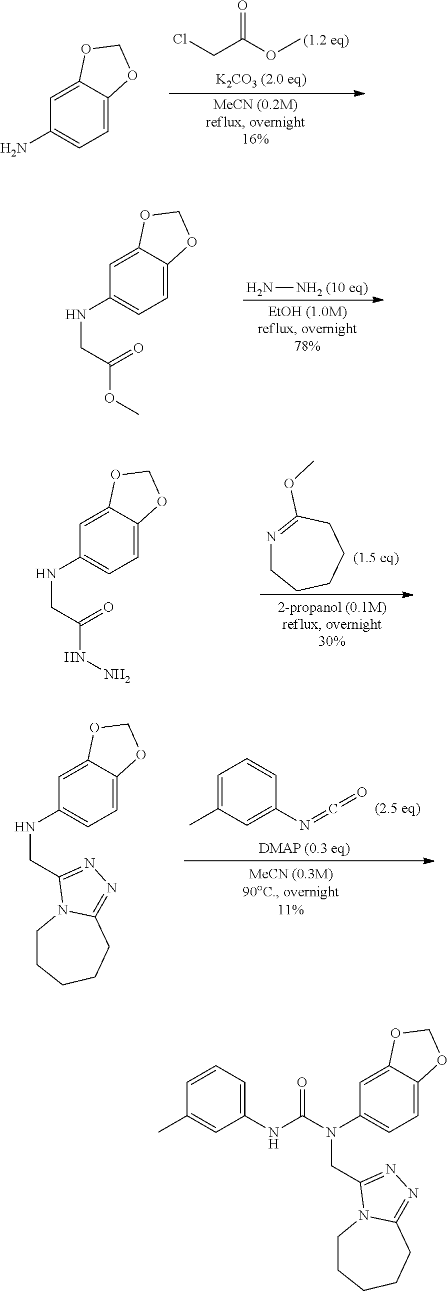

### Step 1-1: Methyl 2-(benzo[d][1,3]dioxol-5-ylamino)acetate

Benzo[d][1,3]dioxol-5-amine (3.0 mL, 30.0 mmol) and K₂CO₃ (8.3 g, 60.0 mmol) were dissolved in 150 mL of MeCN. The flask was cooled on ice for 10 minutes while the solution was stirred under an N₂ atmosphere. Chloroacetyl chloride (3.0 mL, 36.0 mmol) was added thereto dropwise under an N₂ atmosphere, and the mixture was stirred overnight. The reaction was terminated by adding water to the mixture, and the reaction mixture was stirred for another 5 minutes. The organic layer was separated, and the aqueous layer was extracted with ethyl acetate (EA). The organic layers were collected, washed with salt water, dried over anhydrous Na₂SO₄, and concentrated in vacuo. Purification was performed using silica gel chromatography (5% MeOH in DCM) to obtain the title compound.

### Step 1-2: 2-(Benzo[d][1,3]dioxol-5-ylamino)acetohydrazide

Hydrazine hydrate (1.2 mL, 38.2 mmol) was added to an ethanol (38 mL, 0.1 M) solution of methyl 2-(benzo[d][1,3]dioxol-5-ylamino)acetate (800 mg, 38.2 mmol) obtained in step 1-1 above, and the mixture was stirred at 80° C. overnight. The reaction mixture was filtered to obtain the title compound.

### Step 1-3: (E)-7-Methoxy-3,4,5,6-tetrahydro-2H-azepine

Azepan-2-one (3.00 g, 26.5 mmol) was dissolved in DCM (133 mL, 0.2 M). Trimethyloxonium tetrafluoroborate (5.9 g, 39.7 mmol) was added to the reaction mixture. The mixture was stirred at room temperature (30° C.) for 24 hours. Thereafter, the mixture was cooled to 0° C., and a saturated aqueous NaHCO₃ solution was gradually added thereto to adjust the pH of the solution to 8.0. Thereafter, the reaction mixture was stirred at room temperature for 30 minutes and then extracted with DCM. The organic layer was washed with salt water, dried over Na₂SO₄, and concentrated in vacuo to obtain the title compound.

### Step 1-4: N-((6,7,8,9-Tetrahydro-5H-[1,2,4]triazolo[4,3-a]azepin-3-yl)methyl)benzo[d][1,3]dioxol-5-amine

To a solution of 2-(benzo[d][1,3]dioxol-5-ylamino)acetohydrazide (1.00 g, 6.0 mmol) obtained in step 1-2 above in 120 mL of 2-propanol, 7-methoxy-3,4,5,6-tetrahydro-2H-azepine (1.14 g, 9.0 mmol) obtained in step 1-3 above was added, and the mixture was refluxed (80° C.). After completion of the reaction, the mixture was cooled, the precipitate was filtered off, and the filtrate was purified to obtain the title compound.

### Step 1-5: 1-(Benzo[d][1,3]dioxol-5-yl)-1-((6,7,8,9-tetrahydro-5H-[1,2,4]triazolo[4,3-a]azepin-3-yl)methyl)-3-m-tolylurea

A solution of N-((6,7,8,9-tetrahydro-5H-[1,2,4]triazolo[4,3-a]azepin-3-yl)methyl)benzo[d][1,3]dioxol-5-amine (100 mg, 0.35 mmol) obtained in step 1-4 above, 1-isocyanato-3-methylbenzene, (0.11 mL, 0.88 mmol), and DMAP (128 mg, 1.05 mmol) in MeCN (1.2 mL, 0.3 M) was stirred at 90° C. overnight. The reaction was terminated with EA/water and evaporated in vacuo. Thereafter, the reaction mixture was purified using silica gel chromatography (MeOH in DCM system), washed with EA, and filtered to obtain the title compound.

LC/MS [M+H]⁺: 421.33

¹H NMR (300 MHz, CDCl₃) δ 7.17 (t, J=7.7 Hz, 1H), 7.11 (s, 2H), 6.84 (d, J=8.1 Hz, 2H), 6.70-6.63 (m, 2H), 6.05 (s, 2H), 5.08 (s, 2H), 4.29-4.22 (m, 2H), 3.05-2.98 (m, 2H), 2.32 (s, 3H), 1.91 (d, J=4.7 Hz, 2H), 1.86-1.72 (m, 4H).

The following compounds of Examples 2 to 47 were synthesized by conducting reactions in a manner similar to that in Example 1 using appropriate reactants in consideration of the structures of the title compounds. In these Examples, reactions for protection and deprotection of functional groups and/or introduction of additional substituents were additionally conducted depending on whether or not reactive sub-stituents were included. Hereinafter, the compounds of Examples 2 to 47, the intermediates thereof, and the data for identifying these compounds are sequentially disclosed.

### Example 2. 1-(Benzo[d][1,3]dioxol-5-yl)-3-(3-chloro-4-fluorophenyl)-1-((6,7,8,9-tetrahydro-5H-[1,2,4]triazolo[4,3-a]azepin-3-yl)methyl)urea

LC/MS [M+H]⁺: 460.28

¹H NMR (300 MHz, CDCl₃) δ 7.47 (dd, J=6.5 Hz, 2.6 Hz, 1H), 7.09 (dd, J=4.2 Hz, 2.6 Hz, 1H), 7.03 (t, J=8.7 Hz, 1H), 6.84 (d, J=8.1 Hz, 1H), 6.73-6.64 (m, 2H), 6.38 (s, 1H), 6.06 (s, 2H), 5.04 (s, 2H), 4.26-4.18 (m, 2H), 3.05-2.97 (m, 2H), 1.91 (d, J=4.8 Hz, 2H), 1.78 (dd, J=11.6 Hz, 6.1 Hz, 4H).

### Example 3. 3-(3-Chloro-4-fluorophenyl)-1-(4-methoxyphenyl)-1-((6,7,8,9-tetrahydro-5H-[1,2,4]triazolo[4,3-a]azepin-3-yl)methyl)urea

### Step 3-1: Methyl 2-(4-methoxyphenylamino)acetate

LC/MS [M+H]⁺: 197

¹H NMR (500 MHz, DMSO-d₆) δ 6.80-6.66 (m, 2H), 6.56-6.45 (m, 2H), 5.58 (t, J=6.6 Hz, 1H), 3.84 (d, J=6.5 Hz, 2H), 3.63 (s, 6H).

### Step 3-2: 2-(4-Methoxyphenylamino)acetohydrazide

LC/MS [M+H]⁺: 197

¹H NMR (300 MHz, DMSO-d₆) δ 9.02 (s, 1H), 6.77-6.66 (m, 2H), 6.57-6.46 (m, 2H), 5.44 (t, J=6.2 Hz, 1H), 4.22 (s, 2H), 3.63 (s, 3H), 3.55 (d, J=6.2 Hz, 2H).

### Step 3-3: 4-Methoxy-N-((6,7,8,9-tetrahydro-5H-[1,2,4]triazolo[4,3-a]azepin-3-yl)methyl)aniline

LC/MS [M+H]⁺: 274

¹H NMR (300 MHz, DMSO-d₆) δ 6.80-6.61 (m, 4H), 5.72 (t, J=5.7 Hz, 1H), 4.28 (d, J=5.7 Hz, 2H), 4.09-3.94 (m, 2H), 3.63 (s, 3H), 2.94-2.77 (m, 2H), 1.87-1.44 (m, 6H).

### Step 3-4: 3-(3-Chloro-4-fluorophenyl)-1-(4-methoxyphenyl)-1-((6,7,8,9-tetrahydro-5H-[1,2,4]triazolo[4,3-a]azepin-3-yl)methyl)urea

LC/MS [M+H]⁺: 446.24

¹H NMR (300 MHz, DMSO) δ 8.01 (s, 1H), 7.68 (d, J=6.8 Hz, 1H), 7.47-7.35 (m, 1H), 7.26 (t, J=9.1 Hz, 1H), 7.16 (d, J=8.6 Hz, 1H), 6.96 (d, J=8.6 Hz, 1H), 4.93 (s, 1H), 4.06 (d, J=4.3 Hz, 1H), 3.78 (s, 2H), 2.83 (d, J=5.1 Hz, 1H), 1.79 (s, 1H), 1.71-1.47 (m, 2H).

### Example 4. 1-(Benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea

### Step 4-1: 5-Methoxy-3,6-dihydro-2H-1,4-oxazine

¹H NMR (300 MHz, CDCl₃) δ 4.05 (s, 2H), 3.74-3.61 (m, 5H), 3.54 (t, J=4.6 Hz, 2H).

### Step 4-2: N-((6,8-dihydro-5H-[1,2,4]triazolo[3,4-c][1,4]oxazin-3-yl)methyl)benzo[d][1,3]dioxol-5-amine

LC/MS [M+H]⁺: 276.1

¹H NMR (300 MHz, CDCl₃) δ 6.68 (d, J=8.3 Hz, 1H), 6.35 (d, J=2.3 Hz, 1H), 6.18 (dd, J=8.3 Hz, 2.4 Hz, 1H), 5.88 (s, 2H), 4.96 (s, 2H), 4.44 (s, 2H), 4.32-3.81 (m, 4H).

### Step 4-3: 2-Chloro-1-fluoro-4-isocyanotobenzene

¹H NMR (300 MHz, CDCl₃) δ 7.18-7.11 (m, 1H), 7.07 (d, J=8.6 Hz, 1H), 6.99-6.93 (m, 1H).

### Step 4-4: 1-(Benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea

LC/MS [M+H]⁺: 448.1

¹H NMR (300 MHz, CDCl₃) δ 7.44 (dd, J=6.5 Hz, 2.4 Hz, 1H), 7.20-6.95 (m, 2H), 6.87-6.76 (m, 3H), 6.29 (s, 1H), 6.06 (s, 2H), 4.98 (s, 4H), 4.38-4.21 (m, 2H), 4.19-3.98 (m, 2H).

### Example 5. 1-(Benzo[d][1,3]dioxol-5-yl)-3-(2-chlorophenyl)-1-((6,7,8,9-tetrahydro-5H-[1,2,4]triazolo[4,3-a]azepin-3-yl)methyl)urea

LC/MS [M+H]⁺: 442.2

¹H NMR (300 MHz, CDCl₃) δ 8.17 (d, J=8.3 Hz, 1H), 7.24-7.20 (m, 1H), 6.98-6.90 (m, 1H), 6.82 (d, J=8.0 Hz, 1H), 6.69-6.64 (m, 1H), 6.01 (s, 2H), 5.06 (s, 2H), 4.38-3.98 (m, 2H), 3.24-2.87 (m, 2H), 1.94-1.84 (m, 2H), 1.84-1.67 (m, 4H).

### Example 6. 3-(3-Chloro-4-fluorophenyl)-1-cyclopentyl-1-((6,7,8,9-tetrahydro-5H-[1,2,4]triazolo[4,3-a]azepin-3-yl)methyl)urea

### Step 6-1: Methyl 2-(cyclopentylamino)acetate

¹H NMR (300 MHz, CDCl₃) δ 3.71 (s, 3H), 3.40 (s, 2H), 3.05 (p, J=6.4 Hz, 1H), 2.23 (d, J=11.8 Hz, 1H), 1.82-1.74 (m, 2H), 1.71-1.62 (m, 2H), 1.57-1.47 (m, 2H), 1.39-1.28 (m, 2H).

### Step 6-2: 2-(Cyclopentylamino)acetohydrazide

¹H NMR (300 MHz, CDCl₃) δ 8.23 (s, 1H), 4.00-3.69 (m, 1H), 3.33 (d, J=12.4 Hz, 2H), 3.13-3.02 (m, 1H), 1.87-1.75 (m, 2H), 1.68 (dt, J=11.2 Hz, 5.8 Hz, 2H), 1.56 (ddt, J=7.5 Hz, 5.0 Hz, 2.9 Hz, 2H), 1.37-1.27 (m, 2H).

### Step 6-3: N-((6,7,8,9-tetrahydro-5H-[1,2,4]triazolo[4,3-a]azepin-3-yl)methyl)cyclopentanamine

¹H NMR (300 MHz, CDCl₃) δ 4.06-3.99 (m, 2H), 3.86 (s, 2H), 3.42-3.34 (m, 2H), 3.13-3.03 (m, 1H), 2.94 (dd, J=7.1 Hz, 4.1 Hz, 2H), 2.40-2.30 (m, 2H), 1.87-1.82 (m, 2H), 1.76-1.71 (m, 4H), 1.63 (dd, J=4.8 Hz, 2.3 Hz, 2H), 1.53-1.51 (m, 2H).

### Step 6-4: 3-(3-Chloro-4-fluorophenyl)-1-cyclopentyl-1-((6,7,8,9-tetrahydro-5H-[1,2,4]triazolo[4,3-a]azepin-3-yl)methyl)urea

LC/MS [M+H]⁺: 408.24

¹H NMR (300 MHz, CDCl₃) δ 8.38 (s, 1H), 7.59 (dd, J=6.6 Hz, 2.6 Hz, 1H), 7.26-7.19 (m, 1H), 7.03 (t, J=8.8 Hz, 1H), 4.56 (s, 2H), 4.34 (t, J=8.3 Hz, 1H), 4.11-4.03 (m, 2H), 3.05-2.97 (m, 2H), 1.91 (d, J=3.7 Hz, 4H), 1.76 (dd, J=11.1 Hz, 6.6 Hz, 6H), 1.67-1.56 (m, 4H).

### Example 7. 1-(Benzo[d][1,3]dioxol-5-yl)-3-(3-chloro-4-fluorophenyl)-1-((5,6,8,9-tetrahydro-[1,2,4]triazolo[4,3-d][1,4]oxazepin-3-yl)methyl)urea

### Step 7-1: (E)-5-Methoxy-2,3,6,7-tetrahydro-1,4-oxazepine

¹H NMR (500 MHz, CDCl₃) δ 3.70-3.67 (m, 2H), 3.65-3.62 (m, 2H), 3.59 (s, 3H), 3.56-3.52 (m, 2H), 2.67-2.62 (m, 2H).

### Step 7-2: N-((5,6,8,9-tetrahydro-[1,2,4]triazolo[4,3-d][1,4]oxazepin-3-yl)methyl)benzo[d][1,3]dioxol-5-amine

¹H NMR (500 MHz, CDCl₃) δ 6.70 (d, J=8.3 Hz, 2H), 6.37 (d, J=2.3 Hz, 2H), 6.20 (dd, J=8.3 Hz, 2.4 Hz, 2H), 5.90 (s, 4H), 4.39 (d, J=5.6 Hz, 4H), 4.22-4.17 (m, 4H), 4.18-4.09 (m, 3H), 3.92-3.85 (m, 8H), 3.26 (dd, J=5.7 Hz, 4.2 Hz, 4H).

### Step 7-3: 1-(Benzo[d][1,3]dioxol-5-yl)-3-(3-chloro-4-fluorophenyl)-1-((5,6,8,9-tetrahydro-[1,2,4]triazolo[4,3-d][1,4]oxazepin-3-yl)methyl)urea

LC/MS [M+H]⁺: 462.17

¹H NMR (300 MHz, DMSO) δ 8.04 (s, 1H), 7.68 (dd, J=6.9 Hz, 2.5 Hz, 1H), 7.46-7.38 (m, 1H), 7.26 (t, J=9.1 Hz, 1H), 6.93 (dd, J=8.8 Hz, 5.1 Hz, 2H), 6.74 (dd, J=8.2 Hz, 2.0 Hz, 1H), 6.07 (s, 2H), 4.92 (s, 2H), 4.22-4.16 (m, 2H), 3.79-3.74 (m, 2H), 3.74-3.67 (m, 2H), 3.09-3.02 (m, 2H).

### Example 8. 1-(Benzo[d][1,3]dioxol-5-yl)-3-(3-cyanophenyl)-1-((6,7,8,9-tetrahydro-5H-[1,2,4]triazolo[4,3-a]azepin-3-yl)methyl)urea

LC/MS [M+H]⁺: 432.2

¹H NMR (300 MHz, CDCl₃) δ 7.77 (s, 1H), 7.42 (dt, J=7.6 Hz, 2.1 Hz, 1H), 7.37-7.27 (m, 2H), 6.83 (d, J=8.1 Hz, 1H), 6.72 (d, J=2.0 Hz, 1H), 6.68 (dd, J=8.1 Hz, 2.1 Hz, 1H), 6.56 (s, 1H), 6.04 (s, 2H), 5.02 (s, 2H), 4.38-4.08 (m, 2H), 3.01-2.97 (m, 2H), 1.99-1.86 (m, 2H), 1.85-1.71 (m, 4H).

### Example 9. 1-(3-Chloro-4-fluorophenyl)-3-(4-methoxyphenyl)-1-methyl-3-((6,7,8,9-tetrahydro-5H-[1,2,4]triazolo[4,3-a]azepin-3-yl)methyl)urea

LC/MS [M+H]⁺: 460.08

¹H NMR (300 MHz, CDCl₃) δ 6.86 (t, J=8.6 Hz, 1H), 6.76-6.73 (m, 1H), 6.72 (s, 1H), 6.70-6.63 (m, 2H), 6.58 (d, J=9.0 Hz, 2H), 4.91 (s, 2H), 4.22-4.07 (m, 2H), 3.72 (s, 3H), 3.12 (s, 3H), 3.03-2.93 (m, 2H), 1.90 (d, J=4.7 Hz, 2H), 1.83-1.63 (m, 6H).

### Example 10. 3-(3-Chloro-4-fluorophenyl)-1-(1H-indol-6-yl)-1-((6,7,8,9-tetrahydro-5H-[1,2,4]triazolo[4,3-a]azepin-3-yl)methyl)urea

### Step 10-1: Methyl 2-(1H-indol-6-ylamino)acetate

LC/MS [M+H]⁺: 206

¹H NMR (300 MHz, DMSO) δ 10.55 (s, 1H), 7.24 (d, J=8.4 Hz, 1H), 7.00 (dd, J=3.0 Hz, 2.4 Hz, 1H), 6.45 (dd, J=8.4 Hz, 2.1 Hz, 1H), 6.40 (d, J=1.9 Hz, 1H), 6.21 (ddd, J=2.9 Hz, 1.9 Hz, 0.7 Hz, 1H), 5.71 (t, J=6.5 Hz, 1H), 3.90 (d, J=6.5 Hz, 2H), 3.65 (s, 3H)

### Step 10-2: 2-(1H-indol-6-ylamino)acetohydrazide

Crude

### Step 10-3: N-((6,7,8,9-tetrahydro-5H-[1,2,4]triazolo[4,3-a]azepin-3-yl)methyl)-1H-indol-6-amine

Crude

### Step 10-4: 3-(3-Chloro-4-fluorophenyl)-1-(1H-indol-6-yl)-1-((6,7,8,9-tetrahydro-5H-[1,2,4]triazolo[4,3-a]azepin-3-yl)methyl)urea

LC/MS [M+H]⁺: 455.22

¹H NMR (300 MHz, DMSO) δ 11.35-11.09 (s, 1H), 7.99 (s, 1H), 7.68 (dd, J=6.9 Hz, 2.5 Hz, 1H), 7.55 (d, J=8.3 Hz, 1H), 7.24 (d, J=2.7 Hz, 1H), 7.40-7.36 (m, J=4.2, 2.8 Hz, 1H), 7.29 (s, 1H), 7.24 (dd, J=9.1 Hz, 1H), 6.86 (dd, J=8.3 Hz, 1.7 Hz, 1H), 6.47 (s, 1H), 5.00 (s, 2H), 4.14-4.02 (m, 2H), 2.93-2.74 (m, 2H), 1.97-1.73 (m, J=3.9 Hz, 2H), 1.67-1.59 (m, 2H), 1.59-1.50 (m, 2H).

### Example 11. 1-(Benzo[d][1,3]dioxol-5-yl)-3-cyclopentyl-1-((6,7,8,9-tetrahydro-5H-[1,2,4]triazolo[4,3-a]azepin-3-yl)methyl)urea

LC/MS [M+H] 398

¹H NMR (300 MHz, Methanol-d₄) δ 6.82 (d, J=8.2 Hz, 1H), 6.65 (d, J=2.1 Hz, 1H), 6.57 (dd, J=8.2 Hz, 2.2 Hz, 1H), 6.01 (s, 2H), 5.00 (s, 2H), 4.31-4.16 (m, 2H), 4.09-3.94 (m, 1H), 3.04-2.90 (m, 2H), 1.99-1.85 (m, 4H), 1.79-1.71 (m, 4H), 1.64-1.54 (m, 4H), 1.41-1.29 (m, 2H).

### Example 12. 3-(3-Chloro-4-fluorophenyl)-1-(4-isopropoxyphenyl)-1-((6,7,8,9-tetrahydro-5H-[1,2,4]triazolo[4,3-a]azepin-3-yl)methyl)urea

### Step 12-1: 1-Isopropoxy-4-nitrobenzene

¹H NMR (300 MHz, CDCl₃) δ 8.23-8.15 (m, 2H), 6.95-6.88 (m, 2H), 4.73-4.60 (m, 1H), 1.39 (d, J=6.1 Hz, 6H).

### Step 12-2: 4-Isopropoxyaniline

LC/MS [M+H]⁺: 152.99

¹H NMR (300 MHz, CDCl₃) δ 6.80-6.69 (m, 2H), 6.68-6.58 (m, 2H), 4.44-4.29 (m, 1H), 3.40 (s, 2H), 1.27 (t, J=5.6 Hz, 6H).

### Step 12-3: Methyl 2-(4-isopropoxyphenylamino)acetate

LC/MS [M+H]⁺: 223.81

¹H NMR (300 MHz, CDCl₃) δ 6.83-6.74 (m, 2H), 6.60-6.52 (m, 2H), 4.37 (hept, J=6.1 Hz, 1H), 4.02 (s, 1H), 3.88 (s, 2H), 3.77 (s, 3H), 1.28 (d, J=6.1 Hz, 6H).

### Step 12-4: 2-(4-Isopropoxyphenylamino)acetohydrazide

LC/MS [M+H]⁺: 225.09

¹H NMR (300 MHz, DMSO) δ 9.02 (s, 1H), 6.73-6.65 (m, 2H), 6.51-6.44 (m, 2H), 5.45 (t, J=6.1 Hz, 1H), 4.34 (dt, J=12.1 Hz, 6.0 Hz, 1H), 4.22 (s, 2H), 3.55 (d, J=6.2 Hz, 2H), 1.18 (d, J=6.0 Hz, 6H).

### Step 12-5: 4-Isopropoxy-N-((6,7,8,9-tetrahydro-5H-[1,2,4]triazolo[4,3-a]azepin-3-yl)methyl)aniline

LC/MS [M+H]⁺: 302.32

¹H NMR (300 MHz, MeOD) δ 6.70 (dd, J=20.8 Hz, 8.9 Hz, 4H), 4.45-4.31 (m, 3H), 4.20-4.12 (m, 2H), 2.99-2.91 (m, 2H), 1.88 (d, J=5.1 Hz, 2H), 1.77-1.63 (m, 4H), 1.23 (d, J=6.0 Hz, 6H).

### Step 12-6: 3-(3-Chloro-4-fluorophenyl)-1-(4-isopropoxyphenyl)-1-((6,7,8,9-tetrahydro-5H-[1,2,4]triazolo[4,3-a]azepin-3-yl)methyl)urea

LC/MS [M+H]⁺: 472.16

¹H NMR (300 MHz, DMSO) δ 8.01 (s, 1H), 7.67 (d, J=5.6 Hz, 1H), 7.39 (s, 1H), 7.26 (t, J=8.9 Hz, 1H), 7.12 (d, J=8.1 Hz, 2H), 6.91 (d, J=8.2 Hz, 2H), 4.92 (s, 2H), 4.60 (dt, J=11.2 Hz, 5.5 Hz, 1H), 4.04 (s, 2H), 2.84 (s, 2H), 1.77 (s, 2H), 1.58 (s, 4H), 1.27 (d, J=5.8 Hz, 6H).

### Example 13. 3-(3-Chloro-4-fluorophenyl)-1-((6,7,8,9-tetrahydro-5H-[1,2,4]triazolo[4,3-a]azepin-3-yl)methyl)-1-(4-(trifluoromethyl)phenyl)urea

### Step 13-1: Methyl 2-(4-(trifluoromethyl)phenylamino)acetate

LC/MS [M+H]⁺: 234.07

¹H NMR (300 MHz, CDCl₃) δ 7.43 (d, J=8.5 Hz, 2H), 6.61 (d, J=8.5 Hz, 2H), 4.61 (s, 1H), 3.95 (d, J=5.1 Hz, 2H), 3.81 (s, 3H).

### Step 13-2: 2-(4-(Trifluoromethyl)phenylamino)acetohydrazide

LC/MS [M+H]⁺: 233.80

¹H NMR (300 MHz, DMSO) δ 9.17 (s, 1H), 7.38 (d, J=8.6 Hz, 2H), 6.64 (t, J=6.8 Hz, 2H), 4.25 (s, 2H), 3.69 (d, J=6.1 Hz, 2H).

### Step 13-3: N-((6,7,8,9-tetrahydro-5H-[1,2,4]triazolo[4,3-a]azepin-3-yl)methyl)-4-(trifluoromethyl)aniline

Crude

### Step 13-4: 3-(3-Chloro-4-fluorophenyl)-1-((6,7,8,9-tetrahydro-5H-[1,2,4]triazolo[4,3-a]azepin-3-yl)methyl)-1-(4-(trifluoromethyl)phenyl)urea

LC/MS [M+H]⁺: 482.02

¹H NMR (300 MHz, CDCl₃) δ 7.72 (d, J=8.3 Hz, 2H), 7.49 (dd, J=8.1 Hz, 5.1 Hz, 3H), 7.11 (dd, J=8.2 Hz, 3.4 Hz, 1H), 7.02 (t, J=8.7 Hz, 1H), 6.66 (s, 1H), 5.05 (s, 2H), 4.17-4.09 (m, 2H), 3.04-2.94 (m, 2H), 1.89 (d, J=4.6 Hz, 2H), 1.81-1.68 (m, 4H).

### Example 14. 3-(3-Chloro-4-fluorophenyl)-1-(4-(methylamino) phenyl)-1-((6,7,8,9-tetrahydro-5H-[1,2,4]triazolo[4,3-a]azepin-3-yl)methyl)urea

### Step 14-1: Methyl 2-(4-(tert-butoxycarbonyl(methyl)amino)phenylamino)acetate

Crude

### Step 14-2: tert-Butyl 4-(2-hydrazinyl-2-oxoethylamino)phenyl(methyl)carbamate

Crude

### Step 14-3: tert-Butyl methyl(4-((6,7,8,9-tetrahydro-5H-[1,2,4]triazolo[4,3-a]azepin-3-yl)methylamino)phenyl)carbamate

Crude

### Step 14-4: tert-Butyl 4-(3-(3-chloro-4-fluorophenyl)-1-((6,7,8,9-tetrahydro-5H-[1,2,4]triazolo[4,3-a]azepin-3-yl)methyl)ureido)phenyl(methyl)carbamate

LC/MS [M+H]⁺: 545.27

### Step 14-5: 3-(3-Chloro-4-fluorophenyl)-1-(4-(methylamino)phenyl)-1-((6,7,8,9-tetrahydro-5H-[1,2,4]triazolo[4,3-a]azepin-3-yl)methyl)urea

LC/MS [M+H]⁺: 445.16

¹H NMR (300 MHz, DMSO) δ 8.32 (s, 1H), 7.70 (m, 1H), 7.40 (n, 1H), 7.28 (m, 3H), 7.01 (m, 2H), 5.14 (s, 2H), 4.29 (m, 2H), 3.70 (m, 1H), 3.50-3.44 (m, 1H), 2.78 (s, 3H), 1.74 (m, 6H).

### Example 15. 3-(3-Chloro-4-fluorophenyl)-1-((4,5-dimethyl-4H-1,2,4-triazol-3-yl)methyl)-1-(4-methoxyphenyl)urea

### Step 15-1: N-((4,5-dimethyl-4H-1,2,4-triazol-3-yl)methyl)benzo[d][1,3]dioxol-5-amine

Crude

### Step 15-2: 3-(3-Chloro-4-fluorophenyl)-1-((4,5-dimethyl-4H-1,2,4-triazol-3-yl)methyl)-1-(4-methoxyphenyl)urea

LC/MS [M+H]⁺: 406.12

¹H NMR (300 MHz, DMSO) δ 8.02 (s, 1H), 7.70 (dd, J=6.9 Hz, 2.5 Hz, 1H), 7.47-7.37 (m, 1H), 7.27 (t, J=9.1 Hz, 1H), 7.24-7.18 (d, 2H), 6.96 (d, J=8.8 Hz, 2H), 4.92 (s, 2H), 3.78 (s, 3H), 3.51 (s, 3H), 2.29 (s, 3H).

### Example 16. 1-(Benzo[d]thiazol-6-yl)-3-(3-chloro-4-fluorophenyl)-1-((6,7,8,9-tetrahydro-5H-[1,2,4]triazolo [4,3-a]azepin-3-yl)methyl)urea

### Step 16-1: Ethyl 2-(benzo[d]thiazol-6-ylamino)acetate

¹H NMR (300 MHz, Chloroform-d) δ 8.69 (s, 1H), 7.91 (d, J=8.8 Hz, 1H), 7.01 (d, J=2.4 Hz, 1H), 6.86 (dd, J=8.9 Hz, 2.4 Hz, 1H), 4.53 (s, 1H), 4.27 (q, J=7.2 Hz, 2H), 3.96 (d, J=4.6 Hz, 2H), 1.31 (t, J=7.1 Hz, 3H).

### Step 16-2: 2-(Benzo[d]thiazol-6-ylamino)acetohydrazide

¹H NMR (300 MHz, DMSO-d₆) δ 9.16 (s, 1H), 7.77 (d, J=8.8 Hz, 1H), 7.05 (d, J=2.3 Hz, 1H), 6.89 (dd, J=8.9 Hz, 2.3 Hz, 1H), 6.28 (t, J=6.1 Hz, 1H).

### Step 16-3: N-((6,7,8,9-tetrahydro-5H-[1,2,4]triazolo[4,3-a]azepin-3-yl)methyl)benzo[d]thiazol-6-amine

¹H NMR (300 MHz, DMSO) δ 8.94 (s, 1H), 7.77 (d, J=8.8 Hz, 1H), 7.31 (d, J=1.8 Hz, 1H), 6.95 (dd, J=8.8 Hz, 2.1 Hz, 1H), 6.54 (t, J=5.3 Hz, 1H), 4.42 (d, J=5.4 Hz, 2H), 4.12-3.98 (m, 2H), 2.96-2.80 (m, 2H), 1.78 (m, J=4.3 Hz, 2H), 1.65 (m, 2H), 1.57 (m, J=5.0 Hz, 2H).

### Step 16-4: 1-(Benzo[d]thiazol-6-yl)-3-(3-chloro-4-fluorophenyl)-1-((6,7,8,9-tetrahydro-5H-[1,2,4]triazolo[4,3-a]azepin-3-yl)methyl)urea

¹H NMR (300 MHz, DMSO) δ 9.44 (s, 1H), 8.27 (s, 1H), 8.17 (d, J=1.8 Hz, 1H), 8.10 (d, J=8.6 Hz, 1H), 7.66 (dd, J=6.9 Hz, 2.5 Hz, 1H), 7.44 (dd, J=8.7 Hz, 1.9 Hz, 1H), 7.38 (dd, J=4.2 Hz, 2.7 Hz, 1H), 7.27 (t, J=9.1 Hz, 1H), 5.06 (s, 2H), 4.21-4.01 (m, 2H), 2.94-2.77 (m, 2H), 1.79 (s, 2H), 1.63 (s, 2H), 1.57 (m, J=10.5 Hz, 2H).

### Example 17. tert-Butyl 5-(3-(3-chloro-4-fluorophenyl)-1-((6,7,8,9-tetrahydro-5H-[1,2,4]triazolo[4,3-a]azepin-3-yl)methyl)ureido)-1H-indole-1-carboxylate

### Step 17-1: tert-Butyl 5-nitro-1H-indol-1-carboxylate

LC/MS [M+H]⁺: 161.16

¹H NMR (300 MHz, CDCl₃) δ 8.49 (d, J=2.2 Hz, 1H), 8.30-8.18 (m, 2H), 7.74 (d, J=3.7 Hz, 1H), 6.72 (d, J=3.7 Hz, 1H), 1.70 (s, 9H).

### Step 17-2: tert-Butyl 5-aminoindolin-1-carboxylate

LC/MS [M+H]⁺: 232.85

¹H NMR (500 MHz, CDCl₃) δ 7.91 (s, 1H), 7.51 (s, 1H), 6.84 (d, J=2.2 Hz, 1H), 6.71 (dd, J=8.7 Hz, 2.3 Hz, 1H), 6.40 (d, J=3.6 Hz, 1H), 3.60 (s, 2H), 1.65 (s, 9H).

### Step 17-3: tert-Butyl 5-(2-methoxy-2-oxoethylamino)-1H-indol-1-carboxylate

LC/MS [M+H]⁺: 305.02

¹H NMR (300 MHz, MeOD) δ 7.86 (d, J=9.4 Hz, 1H), 7.48 (d, J=3.7 Hz, 1H), 6.70 (dd, J=7.9 Hz, 2.1 Hz, 2H), 6.43 (d, J=3.6 Hz, 1H), 3.95 (s, 2H), 3.73 (s, 3H), 1.65 (s, 9H).

### Step 17-4: tert-Butyl 5-(2-hydrazinyl-2-oxoethylamino)-1H-indol-1-carboxylate

Crude

### Step 17-5: tert-Butyl 5-((6,7,8,9-tetrahydro-5H-[1,2,4]triazolo[4,3-a]azepin-3-yl)methylamino)-1H-indol-1-carboxylate

¹H NMR (300 MHz, Chloroform-d) δ 7.94 (d, J=7.8 Hz, 1H), 7.52 (d, J=3.1 Hz, 1H), 6.90 (s, 1H), 6.75 (d, J=8.8 Hz, 1H), 6.45 (d, J=3.6 Hz, 1H), 4.07-3.97 (m, 2H), 3.04-2.95 (m, 2H), 1.93-1.84 (m, 2H), 1.74 (s, 5H), 1.65 (s, 9H).

### Step 17-6: tert-Butyl 5-(3-(3-chloro-4-fluorophenyl)-1-((6,7,8,9-tetrahydro-5H-[1,2,4]triazolo[4,3-a]azepin-3-yl)methyl)ureido)-1H-indole-1-carboxylate

LC/MS [M-H]⁻: 551.28

¹H NMR (300 MHz, CDCl₃) δ 8.20 (d, J=9.0 Hz, 1H), 7.67 (d, J=3.6 Hz, 1H), 7.47 (d, J=1.8 Hz, 1H), 7.42 (dd, J=6.5 Hz, 2.4 Hz, 1H), 7.12-7.06 (m, 1H), 7.01 (dt, J=17.3 Hz, 6.7 Hz, 2H), 6.57 (d, J=3.7 Hz, 1H), 6.30 (s, 1H), 5.12 (s, 2H), 4.23 (d, J=4.8 Hz, 2H), 3.03-2.94 (m, 2H), 1.89 (s, 2H), 1.77 (d, J=22.9 Hz, 4H), 1.68 (s, 9H).

### Example 18. 3-(3-Chloro-4-fluorophenyl)-1-(1H-indol-5-yl)-1-((6,7,8,9-tetrahydro-5H-[1,2,4]triazolo[4,3-a]azepin-3-yl)methyl)urea

### Step 18-1: tert-Butyl 5-nitro-1H-indol-1-carboxylate

¹H NMR (300 MHz, CDCl₃) δ 8.49 (d, J=2.2 Hz, 1H), 8.30-8.18 (m, 2H), 7.74 (d, J=3.7 Hz, 1H), 6.72 (d, J=3.7 Hz, 1H), 1.70 (s, 9H).

### Step 18-2: tert-Butyl 5-aminoindolin-1-carboxylate

LC/MS [M+H]⁺: 234.81

¹H NMR (300 MHz, DMSO) δ 7.17 (d, J=88.3 Hz, 1H), 6.38 (s, 1H), 6.28 (dd, J=8.5 Hz, 2.2 Hz, 1H), 4.66 (s, 2H), 3.74 (t, J=8.6 Hz, 2H), 2.85 (t, J=8.5 Hz, 2H), 1.41 (s, 9H).

### Step 18-3: tert-Butyl 5-(2-methoxy-2-oxoethylamino)-1H-indol-1-carboxylate

LC/MS [M+H]⁺: 305.02

¹H NMR (300 MHz, MeOD) δ 7.86 (d, J=9.4 Hz, 1H), 7.48 (d, J=3.7 Hz, 1H), 6.70 (dd, J=7.9 Hz, 2.1 Hz, 2H), 6.43 (d, J=3.6 Hz, 1H), 3.95 (s, 2H), 3.73 (s, 3H), 1.65 (s, 9H).

### Step 18-4: tert-Butyl 5-(2-hydrazinyl-2-oxoethylamino)-1H-indol-1-carboxylate

Crude

### Step 18-5: tert-Butyl 5-((6,7,8,9-tetrahydro-5H-[1,2,4]triazolo[4,3-a]azepin-3-yl)methylamino)-1H-indol-1-carboxylate

¹H NMR (300 MHz, Chloroform-d) δ 7.94 (d, J=7.8 Hz, 1H), 7.52 (d, J=3.1 Hz, 1H), 6.90 (s, 1H), 6.75 (d, J=8.8 Hz, 1H), 6.45 (d, J=3.6 Hz, 1H), 4.07-3.97 (m, 2H), 3.04-2.95 (m, 2H), 1.93-1.84 (m, 2H), 1.74 (s, 5H), 1.65 (s, 9H).

### Step 18-6: tert-Butyl 5-(3-(3-chloro-4-fluorophenyl)-1-((6,7,8,9-tetrahydro-5H-[1,2,4]triazolo[4,3-a]azepin-3-yl)methyl)ureido)-1H-indol-1-carboxylate

LC/MS [M

⁻:551.28

¹H NMR (300 MHz, CDCl₃) δ 8.20 (d, J=9.0 Hz, 1H), 7.67 (d, J=3.6 Hz, 1H), 7.47 (d, J=1.8 Hz, 1H), 7.42 (dd, J=6.5 Hz, 2.4 Hz, 1H), 7.12-7.06 (m, 1H), 7.01 (dt, J=17.3 Hz, 6.7 Hz, 2H), 6.57 (d, J=3.7 Hz, 1H), 6.30 (s, 1H), 5.12 (s, 2H), 4.23 (d, J=4.8 Hz, 2H), 3.03-2.94 (m, 2H), 1.89 (s, 2H), 1.77 (d, J=22.9 Hz, 4H), 1.68 (s, 9H).

### Step 18-7: 3-(3-Chloro-4-fluorophenyl)-1-(1H-indol-5-yl)-1-((6,7,8,9-tetrahydro-5H-[1,2,4]triazolo[4,3-a]azepin-3-yl)methyl)urea

LC/MS [M+H]⁺: 453.06

¹H NMR (300 MHz, DMSO) δ 11.24 (s, 1H), 7.86 (s, 1H), 7.69-7.63 (m, 1H), 7.39 (d, J=10.3 Hz, 4H), 7.22 (t, J=9.1 Hz, 1H), 6.89 (d, J=8.4 Hz, 1H), 6.44 (s, 1H), 4.98 (s, 2H), 4.06 (s, 2H), 2.81 (s, 2H), 1.77 (s, 2H), 1.58 (s, 4H).

### Example 19. 3-(3-Chloro-4-fluorophenyl)-1-(3-cyanophenyl)-1-((6,7,8,9-tetrahydro-5H-[1,2,4]triazolo[4,3-a]azepin-3-yl)methyl)urea

### Step 19-1: Ethyl 2-(3-cyanophenylamino)acetate

¹H NMR (300 MHz, CDCl₃) δ 7.25 (s, 1H), 7.04 (d, J=7.6 Hz, 1H), 6.86-6.79 (m, 2H), 4.56 (s, 1H), 4.29 (q, J=7.1 Hz, 2H), 3.92 (d, J=5.3 Hz, 2H), 1.34 (t, J=7.1 Hz, 3H).

### Step 19-2: 2-(3-Cyanophenylamino)acetohydrazide

¹H NMR (300 MHz, DMSO) δ 9.17 (s, 2H), 7.26 (t, J=7.8 Hz, 2H), 6.91 (dd, J=23.0 Hz, 8.5 Hz, 4H), 6.45 (t, J=6.1 Hz, 2H), 4.26 (s, 3H), 3.67 (d, J=6.2 Hz, 4H).

### Step 19-3: 3-((6,7,8,9-tetrahydro-5H-[1,2,4]triazolo[4,3-a]azepin-3-yl)methylamino)benzonitrile

¹H NMR (300 MHz, DMSO) δ 7.27 (t, J=7.8 Hz, 1H), 7.08-6.95 (m, 3H), 6.71 (s, 1H), 4.40 (d, J=5.5 Hz, 2H), 4.03-3.98 (m, 2H), 2.90-2.82 (m, 2H), 1.79 (d, J=4.6 Hz, 2H), 1.60 (dd, J=14.4 Hz, 8.3 Hz, 4H).

### Step 19-4: 3-(3-Chloro-4-fluorophenyl)-1-(3-cyanophenyl)-1-((6,7,8,9-tetrahydro-5H-[1,2,4]triazolo[4,3-a]azepin-3-yl)methyl)urea

LC/MS [M+H]⁺: 441.11

¹H NMR (300 MHz, CDCl₃) δ 7.70 (d, J=6.9 Hz, 3H), 7.64 (d, J=6.5 Hz, 1H), 7.50 (dd, J=6.4 Hz, 2.5 Hz, 1H), 7.16-7.02 (m, 2H), 6.72 (s, 1H), 5.04 (s, 2H), 4.16-4.06 (m, 2H), 3.06-2.99 (m, 2H), 1.92 (s, 2H), 1.77 (d, J=5.1 Hz, 4H).

### Example 20. 1-(Benzo[d][1,3]dioxol-5-yl)-3-(6-methylpyridin-2-yl)-1-((6,7,8,9-tetrahydro-5H-[1,2,4]triazolo[4,3-a]azepin-3-yl)methyl)urea

### Step 20-1: N-(6-methylpyridin-2-yl)-1H-imidazol-1-carboxamide

Crude

### Step 20-2: 1-(Benzo[d][1,3]dioxol-5-yl)-3-(6-methylpyridin-2-yl)-1-((6,7,8,9-tetrahydro-5H-[1,2,4]triazolo[4,3-a]azepin-3-yl)methyl)urea

LC/MS [M+H]⁺: 422.3

¹H NMR (300 MHz, CDCl₃) δ 7.86 (d, J=8.3 Hz, 1H), 7.54 (t, J=7.9 Hz, 1H), 6.95 (s, 1H), 6.80 (dd, J=7.7 Hz, 4.4 Hz, 1H), 6.63 (dt, J=8.0 Hz, 2.0 Hz, 1H), 6.03 (s, 1H), 5.06 (s, 1H), 4.32-4.13 (m, 1H), 3.08-2.92 (m, 1H), 2.35 (s, 2H), 1.88-1.87 (m, 2H), 1.83-1.69 (m, 3H).

### Example 21. 3-(3-Chloro-4-fluorophenyl)-1-((6,7,8,9-tetrahydro-5H-[1,2,4]triazolo[4,3-a]azepin-3-yl)methyl)-1-(3-(trifluoromethyl)phenyl)urea

### Step 21-1: Ethyl 2-(3-(trifluoromethyl)phenylamino)acetate

LC/MS [M+H]⁺: 248.99

¹H NMR (500 MHz, CDCl₃) δ 7.29 (d, J=7.9 Hz, 1H), 6.99 (d, J=7.7 Hz, 1H), 6.79 (s, 1H), 6.76 (d, J=8.2 Hz, 1H), 4.50 (s, 1H), 4.27 (q, J=7.1 Hz, 2H), 3.92 (d, J=5.3 Hz, 2H), 1.31 (t, J=7.1 Hz, 3H).

### Step 21-2: 2-(3-(Trifluoromethyl)phenylamino)acetohydrazide

LC/MS [M+H]⁺: 234.95

¹H NMR (300 MHz, CDCl₃) δ 7.67 (s, 1H), 7.31 (t, J=7.9 Hz, 1H), 7.05 (d, J=7.7 Hz, 1H), 6.81 (s, 1H), 6.79-6.71 (m, 1H), 4.46 (s, 1H), 3.89 (d, J=4.7 Hz, 2H), 3.84-3.30 (m, 2H).

### Step 21-3: N-((6,7,8,9-tetrahydro-5H-[1,2,4]triazolo[4,3-a]azepin-3-yl)methyl)-3-(trifluoromethyl)aniline

LC/MS [M+H]⁺: 312.11

¹H NMR (300 MHz, MeOD) δ 7.27 (t, J=7.7 Hz, 1H), 6.98-6.87 (m, 3H), 4.51 (s, 2H), 4.20-4.11 (m, 2H), 3.01-2.91 (m, 2H), 1.89 (d, J=4.9 Hz, 2H), 1.81-1.63 (m, 4H).

### Step 21-4: 3-(3-Chloro-4-fluorophenyl)-1-((6,7,8,9-tetrahydro-5H-[1,2,4]triazolo[4,3-a]azepin-3-yl)methyl)-1-(3-(trifluoromethyl)phenyl)urea

LC/MS [M+H]⁺: 483.98

¹H NMR (300 MHz, CDCl₃) δ 7.68-7.56 (m, 4H), 7.48 (dd, J=6.4 Hz, 2.4 Hz, 1H), 7.06 (ddd, J=22.2 Hz, 13.1 Hz, 6.4 Hz, 2H), 6.72 (s, 1H), 5.04 (s, 2H), 4.15-4.05 (m, 2H), 3.04-2.93 (m, 2H), 1.88 (d, J=4.4 Hz, 2H), 1.74 (d, J=5.0 Hz, 5H).

### Example 22. 3-(3-Chloro-4-fluorophenyl)-1-(3,4-difluorophenyl)-1-((6,7,8,9-tetrahydro-5H-[1,2,4]triazolo[4,3-a]azepin-3-yl)methyl)urea

### Step 22-1: Methyl 2-(3,4-difluorophenylamino)acetate

LC/MS [M+H]⁺: 203

¹H NMR (300 MHz, Chloroform-d) δ 7.00 (q, J=9 Hz, 1H), 6.51-6.38 (m, 1H), 6.35-6.24 (m, 1H), 4.27 (s, 1H), 3.88 (s, 2H), 3.82 (s, 3H).

### Step 22-2: 2-(3,4-Difluorophenylamino)acetohydrazide

LC/MS [M+H] 202

¹H NMR (300 MHz, Chloroform-d) δ 7.60 (s, 1H), 7.03 (q, J=8.9 Hz, 1H), 6.46-6.39 (m, 1H), 6.36-6.24 (m, 1H), 4.20 (s, 1H), 3.91 (s, 2H), 3.84 (d, J=5.5 Hz, 2H).

### Step 22-3: 3,4-Difluoro-N-((6,7,8,9-tetrahydro-5H-[1,2,4]triazolo[4,3-a]azepin-3-yl)methyl)aniline

LC/MS [M+H] 279

¹H NMR (300 MHz, Chloroform-d) δ 7.02 (q, J=9.0 Hz, 1H), 6.55 (ddd, J=12.3 Hz, 6.5 Hz, 2.8 Hz, 1H), 6.49-6.35 (m, 1H), 4.36 (d, J=4.6 Hz, 2H), 4.32 (s, 1H), 4.06-3.93 (m, 2H), 3.10-2.96 (m, 2H), 1.92 (d, J=4.4 Hz, 2H), 1.87-1.67 (m, 4H).

### Step 22-4: 3-(3-Chloro-4-fluorophenyl)-1-(3,4-difluorophenyl)-1-((6,7,8,9-tetrahydro-5H-[1,2,4]triazolo[4,3-a]azepin-3-yl)methyl)urea

LC/MS [M+H] 450

¹H NMR (300 MHz, Methanol-d₄) δ 7.60 (dd, J=6.7 Hz, 2.6 Hz, 1H), 7.43-7.32 (m, 2H), 7.31-7.23 (m, 1H), 7.13 (t, J=9.0 Hz, 2H), 5.09 (s, 2H), 4.33-4.18 (m, 2H), 3.04-2.91 (m, 2H), 2.00-1.89 (m, 2H), 1.88-1.64 (m, 4H).

### Example 23. 3-(3-Chloro-4-fluorophenyl)-1-(4-methoxyphenyl)-1-(1-(6,7,8,9-tetrahydro-5H-[1,2,4]triazolo[4,3-a]azepin-3-yl)ethyl)urea

### Step 23-1: Methyl 2-(4-methoxyphenylamino)propanoate

¹H NMR (300 MHz, CDCl₃) δ 6.79 (d, J=8.91 Hz, 2H), 6.61 (d, J=8.91 Hz, 2H), 4.12 (q, J=6.996 Hz, 1H), 3.75 (s, 3H), 3.72 (s, 3H), 1.47 (d, J=6.93 Hz, 3H)

### Step 23-2: 2-(4-methoxyphenylamino)propanhydrazide

¹H NMR (300 MHz, CDCl₃) δ 6.79 (d, J=8.91 Hz, 2H), 6.61 (d, J=8.91 Hz, 2H), 4.12 (q, J=6.996 Hz, 1H), 3.75 (s, 3H), 3.72 (s, 3H), 1.47 (d, J=6.93 Hz, 3H)

### Step 23-3: 4-Methoxy-N-(1-(6,7,8,9-tetrahydro-5H-[1,2,4]triazolo[4,3-a]azepin-3-yl)ethyl)aniline

¹H NMR (300 MHz, CDCl₃) δ 6.70 (d, J=8.97 Hz, 2H), 6.61 (d, J=9.00 Hz, 2H), 5.58 (d, J=7.95 Hz, 1H), 4.75 (m, 1H), 4.06 (m, 2H), 3.62 (s, 3H), 2.85 (m, 2H), 1.75 (m, 2H), 1.55 (m, 4H), 1.49 (d, J=6.69 Hz, 3H)

### Step 23-4: 3-(3-Chloro-4-fluorophenyl)-1-(4-methoxyphenyl)-1-(1-(6,7,8,9-tetrahydro-5H-[1,2,4]triazolo[4,3-a]azepin-3-yl)ethyl)urea

¹H NMR (300 MHz, DMSO) δ 7.67 (m, 2H), 7.42 (m, 1H), 7.28 (t, J=9.12 Hz, 1H), 6.91 (m, 2H), 6.74 (m, 1H), 6.04 (q, J=6.99 Hz, 1H), 4.15 (m, 2H), 3.77 (s, 3H), 2.91 (m, 2H), 2.09 (s, 3H), 1.82 (m, 6H), 1.40 (d, J=6.87 Hz, 3H)

### Example 24. 3-(3-Chloro-4-fluorophenyl)-1-(4-methoxyphenyl)-1-((4-methyl-5-phenyl-4H-1,2,4-triazol-3-yl)methyl)urea

### Step 24-1: (Z)-methyl N-methylbenzimidate

¹H NMR (300 MHz, CDCl₃) δ 7.54-7.27 (m, 5H), 3.81 (s, 3H), 3.10 (s, 3H).

### Step 24-2: 4-Methoxy-N-((4-methyl-5-phenyl-4H-1,2,4-triazol-3-yl)methyl)aniline

LC/MS [M+H]⁺: 296.2

¹H NMR (300 MHz, CDCl₃) δ 7.77-7.58 (m, 2H), 7.57-7.47 (m, 3H), 6.80 (td, J=9.1 Hz, 2.3 Hz, 4H), 4.50 (d, J=5.7 Hz, 2H), 4.17-3.90 (m, 1H), 3.76 (s, 3H), 3.71 (s, 3H).

### Step 24-3: 3-(3-Chloro-4-fluorophenyl)-1-(4-methoxyphenyl)-1-((4-methyl-5-phenyl-4H-1,2,4-triazol-3-yl)methyl)urea

LC/MS [M+H]⁺: 468.2

¹H NMR (300 MHz, CDCl₃) δ 7.66-7.63 (m, 2H), 7.56-7.48 (m, 3H), 7.46 (dd, J=6.5 Hz, 2.5 Hz, 1H), 7.30-7.27 (m, 2H), 7.14-6.90 (m, 4H), 6.30 (s, 1H), 5.12 (s, 2H), 3.84 (s, 3H) 3.82 (s, 3H).

### Example 25. 3-(3-Chloro-4-fluorophenyl)-1-(4-methoxyphenyl)-1-((5-methyl-4-phenyl-4H-1,2,4-triazol-3-yl)methyl)urea

### Step 25-1: (Z)-methyl N-phenylacetimidate

¹H NMR (300 MHz, CDCl₃) δ 7.28 (dd, J=10.7 Hz, 4.9 Hz, 2H), 7.03 (t, J=7.4 Hz, 1H), 6.85-6.64 (m, 2H), 3.79 (s, 3H), 1.82 (s, 3H).

### Step 25-2: 4-Methoxy-N-((5-methyl-4-phenyl-4H-1,2,4-triazol-3-yl)methyl)aniline

¹H NMR (300 MHz, CDCl₃) δ 7.65-7.48 (m, 3H), 7.26-7.21 (m, 2H), 6.73 (d, J=8.9 Hz, 2H), 6.52 (d, J=8.9 Hz, 2H), 4.24 (d, J=5.6 Hz, 2H), 3.72 (s, 3H), 2.27 (s, 3H)

### Step 25-3: 3-(3-Chloro-4-fluorophenyl)-1-(4-methoxyphenyl)-1-((5-methyl-4-phenyl-4H-1,2,4-triazol-3-yl)methyl)urea

LC/MS [M+H]⁺: 468.2

¹H NMR (300 MHz, CDCl₃) δ 7.57-7.46 (m, 3H), 7.37-7.33 (m, 3H), 7.23-7.20 (m, 2H), 6.99 (m, 4H), 6.31 (s, 1H), 4.81 (s, 2H), 3.84 (s, 3H), 2.27 (s, 3H).

### Example 26. 3-(3-Chloro-4-fluorophenyl)-1-((4-isopropyl-5-methyl-4H-1,2,4-triazol-3-yl)methyl)-1-(4-methoxyphenyl)urea

### Step 26-1: N-isopropylacetamide

¹H NMR (300 MHz, Methanol-d₄) δ 3.93 (hept, J=6.5 Hz, 1H), 1.89 (s, 3H), 1.12 (d, J=6.6 Hz, 6H).

### Step 26-2: (E)-methyl N-isopropylacetimidate

¹H NMR (500 MHz, Chloroform-d) δ 3.59 (s, 3H), 3.48 (p, J=6.3 Hz, 1H), 1.86 (s, 3H), 1.09 (d, J=6.3 Hz, 6H).

### Step 26-3: N-((4-isopropyl-5-methyl-4H-1,2,4-triazol-3-yl)methyl)-4-methoxyaniline

LC/MS [M+H]⁺: 261.95

¹H NMR (300 MHz, Chloroform-d) δ 6.81 (d, J=8.8 Hz, 2H), 6.71 (d, J=8.9 Hz, 2H), 4.55 (hept, J=6.9 Hz, 1H), 4.39 (s, 2H), 3.76 (s, 3H), 2.53 (s, 3H), 1.51 (d, J=7.0 Hz, 6H).

### Step 26-4: 3-(3-Chloro-4-fluorophenyl)-1-((4-isopropyl-5-methyl-4H-1,2,4-triazol-3-yl)methyl)-1-(4-methoxyphenyl)urea

LC/MS [M+H]⁺: 433.96

¹H NMR (300 MHz, Chloroform-d) δ 7.45 (dd, J=6.5 Hz, 2.5 Hz, 1H), 7.18-7.11 (m, 2H), 7.07 (ddd, J=8.9 Hz, 4.2 Hz, 2.7 Hz, 1H), 7.00 (t, J=8.7 Hz, 1H), 6.96-6.90 (m, 2H), 6.34 (s, 1H), 5.08 (s, 2H), 4.87 (hept, J=7.1 Hz, 1H), 3.82 (s, 3H), 2.55 (s, 3H), 1.52 (d, J=7.0 Hz, 6H).

### Example 27. 3-(3-Chloro-4-fluorophenyl)-1-(4-methoxyphenyl)-1-((5,6,8,9-tetrahydro-[1,2,4]triazolo[4,3-d][1,4]oxazepin-3-yl)methyl)urea

### Step 27-1: (E)-5-Methoxy-2,3,6,7-tetrahydro-1,4-oxazepine

¹H NMR (500 MHz, CDCl₃) δ 3.70-3.67 (m, 2H), 3.65-3.62 (m, 2H), 3.59 (s, 3H), 3.56-3.52 (m, 2H), 2.67-2.62 (m, 2H).

### Step 27-2: 4-Methoxy-N-((5,6,8,9-tetrahydro-[1,2,4]triazolo[4,3-d][1,4]oxazepin-3-yl)methyl)aniline

LC/MS [M+H]⁺: 276.13

¹H NMR (300 MHz, Methanol-d₄) δ 6.77-6.71 (m, 2H), 6.68 (d, J=9.1 Hz, 2H), 4.41 (s, 2H), 4.34-4.24 (m, 2H), 3.86-3.76 (m, 4H), 3.68 (s, 3H), 3.19-3.11 (m, 2H).

### Step 27-3: 3-(3-Chloro-4-fluorophenyl)-1-(4-methoxyphenyl)-1-((5,6,8,9-tetrahydro-[1,2,4]triazolo[4,3-d][1,4]oxazepin-3-yl)methyl)urea

LC/MS [M+H]⁺: 448.13

¹H NMR (300 MHz, Chloroform-d) δ 7.41 (dd, J=6.4 Hz, 2.2 Hz, 1H), 7.17 (d, J=8.8 Hz, 2H), 7.09-6.99 (m, 2H), 6.96 (d, J=8.8 Hz, 2H), 6.24 (s, 1H), 5.00 (s, 2H), 4.47-4.36 (m, 2H), 3.95-3.85 (m, 4H), 3.83 (s, 3H), 3.30-3.18 (m, 2H).

### Example 28. 3-(3-Chloro-4-fluorophenyl)-1-(4-cyanophenyl)-1-((6,7,8,9-tetrahydro-5H-[1,2,4]triazolo[4,3-a]azepin-3-yl)methyl)urea

### Step 28-1: Methyl 2-(4-cyanophenylamino)acetate

¹H NMR (300 MHz, CDCl₃) δ 7.52-7.40 (d, 2H), 6.62-6.51 (d, 2H), 4.82 (s, 1H), 4.28 (q, J=7.1 Hz, 2H), 3.92 (d, J=5.1 Hz, 2H), 1.31 (t, J=7.1 Hz, 3H).

### Step 28-2: 2-(4-Cyanophenylamino)acetohydrazide

¹H NMR (300 MHz, DMSO) δ 9.19 (s, 1H), 7.47 (d, J=8.7 Hz, 2H), 6.93 (t, J=6.0 Hz, 1H), 6.64 (d, J=7.4 Hz, 2H), 4.26 (s, 2H), 3.71 (d, J=6.1 Hz, 2H).

### Step 28-3: 4-((6,7,8,9-tetrahydro-5H-[1,2,4]triazolo[4,3-a]azepin-3-yl)methylamino)benzonitrile

¹H NMR (300 MHz, DMSO-d₆) δ 7.56-7.42 (m, 2H), 7.19 (t, J=5.5 Hz, 2H), 6.85-6.74 (m, 2H), 4.44 (d, J=5.5 Hz, 2H), 4.06-3.93 (m, 2H), 2.95-2.77 (m, 2H), 1.58 (dtd, J=25.1 Hz, 13.2 Hz, 11.3 Hz, 6.9 Hz, 6H).

### Step 28-4: 3-(3-Chloro-4-fluorophenyl)-1-(4-cyanophenyl)-1-((6,7,8,9-tetrahydro-5H-[1,2,4]triazolo[4,3-a]azepin-3-yl)methyl)urea

¹H NMR (300 MHz, DMSO) δ 8.75 (s, 1H), 7.88 (d, J=7.5 Hz, 2H), 7.67 (d, J=4.5 Hz, 1H), 7.58 (d, J=7.6 Hz, 2H), 7.46-7.21 (m, 2H), 5.07 (s, 2H), 4.07 (s, 2H), 2.84 (s, 2H), 1.78 (s, 2H), 1.60 (m, J=25.2 Hz, 4H).

### Example 29. 3-(3-Chloro-4-fluorophenyl)-1-((5-isopropyl-4-methyl-4H-1,2,4-triazol-3-yl)methyl)-1-(4-methoxyphenyl)urea

### Step 29-1: N-methylisobutyramide

¹H NMR (500 MHz, Chloroform-d) δ 5.87 (s, 1H), 2.80 (d, J=4.8 Hz, 3H), 2.38 (hept, J=6.9 Hz, 1H), 1.15 (d, J=6.9 Hz, 6H).

### Step 29-2: (E)-methyl N-methylisobutyrimidate

¹H NMR (300 MHz, Chloroform-d) δ 3.58 (s, 3H), 3.03 (s, 3H), 2.93 (p, J=6.9 Hz, 1H), 1.09 (d, J=6.9 Hz, 6H).

### Step 29-3: N-((5-isopropyl-4-methyl-4H-1,2,4-triazol-3-yl)methyl)-4-methoxyaniline

LC/MS [M+H]⁺: 262.08

¹H NMR (300 MHz, Methanol-d₄) δ 6.74 (d, J=9.2 Hz, 2H), 6.69 (d, J=9.2 Hz, 2H), 4.41 (s, 2H), 3.69 (s, 3H), 3.66 (s, 3H), 3.13 (dq, J=13.8 Hz, 6.8 Hz, 1H), 1.33 (d, J=6.9 Hz, 6H).

### Step 29-4: 3-(3-Chloro-4-fluorophenyl)-1-((5-isopropyl-4-methyl-4H-1,2,4-triazol-3-yl)methyl)-1-(4-methoxyphenyl)ure

LC/MS [M+H]⁺: 434.16

¹H NMR (300 MHz, Chloroform-d) δ 7.45 (dd, J=6.5 Hz, 2.5 Hz, 1H), 7.23-7.16 (m, 2H), 7.09-7.03 (m, 1H), 7.00 (t, J=8.7 Hz, 1H), 6.93 (d, J=8.9 Hz, 2H), 6.31 (s, 1H), 5.03 (s, 2H), 3.83 (s, 3H), 3.70 (s, 3H), 3.00 (hept, J=6.7 Hz, 1H), 1.39 (d, J=6.9 Hz, 6H).

### Example 30. 1-(Benzo[d][1,3]dioxol-5-yl)-3-(3-chloro-4-fluorophenyl)-1-((5,6,7,8,9,10-hexahydro-[1,2,4]triazolo[4,3-a]azocin-3-yl)methyl)urea

### Step 30-1: (E)-8-Methoxy-2,3,4,5,6,7-hexahydroazocine

¹H NMR (300 MHz, CDCl₃) δ 3.63 (s, 3H), 3.56-3.33 (m, 2H), 2.44-2.19 (m, 2H), 1.77-1.54 (m, 4H), 1.57-1.28 (m, 4H).

### Step 30-2: N-((5,6,7,8,9,10-hexahydro-[1,2,4]triazolo[4,3-a]azocin-3-yl)methyl)benzo[d][1,3]dioxol-5-amine

Crude

### Step 30-3: 1-(Benzo[d][1,3]dioxol-5-yl)-3-(3-chloro-4-fluorophenyl)-1-((5,6,7,8,9,10-hexahydro-[1,2,4]triazolo[4,3-a]azocin-3-yl)methyl)urea

LC/MS [M+H]⁺: 474.0

¹H NMR (300 MHz, CDCl₃) δ 7.46 (dd, J=6.5 Hz, 2.4 Hz, 1H), 7.15-7.05 (m, 1H), 7.00 (t, J=8.7 Hz, 1H), 6.80 (d, J=8.1 Hz, 1H), 6.76-6.63 (m, 2H), 6.50 (s, 1H), 6.02 (s, 2H), 5.01 (s, 2H), 4.41-4.08 (m, 2H), 3.06-2.75 (m, 2H), 1.93-1.68 (m, 4H), 1.52 (s, 2H), 1.41-1.20 (m, 2H).

### Example 31. 3-(3-Chloro-4-fluorophenyl)-1-((5-cyclopentyl-4-methyl-4H-1,2,4-triazol-3-yl)methyl)-1-(4-methoxyphenyl)urea

### Step 31-1: N-methylcyclopentancarboxamide

¹H NMR (300 MHz, CDCl₃) δ 5.47 (s, 1H), 2.83 (d, J=4.8 Hz, 3H), 2.50 (dd, J=16.0 Hz, 7.9 Hz, 1H), 1.91-1.73 (m, 6H), 1.59 (d, J=2.7 Hz, 2H).

### Step 31-2: (Z)-methyl N-methylcyclopentancarbimidate

¹H NMR (300 MHz, CDCl₃) δ 3.58 (s, 12H), 3.03 (s, 13H), 2.98 (dd, J=10.0 Hz, 5.9 Hz, 4H), 1.81-1.71 (m, 24H), 1.56 (dd, J=6.0 Hz, 2.9 Hz, 10H).

### Step 31-3: N-((5-cyclopentyl-4-methyl-4H-1,2,4-triazol-3-yl)methyl)-4-methoxyaniline

¹H NMR (300 MHz, DMSO) δ 6.76-6.64 (m, 7H), 4.29 (d, J=5.6 Hz, 3H), 3.63 (s, 6H), 3.56 (d, J=7.6 Hz, 5H), 3.22-3.06 (m, 2H), 2.04-1.92 (m, 4H), 1.92-1.91 (m, 1H), 1.84-1.62 (m, 11H).

### Step 31-4: 3-(3-Chloro-4-fluorophenyl)-1-((5-cyclopentyl-4-methyl-4H-1,2,4-triazol-3-yl)methyl)-1-(4-methoxyphenyl)urea

LC/MS [M+H]⁺: 460.08

¹H NMR (300 MHz, CDCl₃) δ 7.46 (dd, J=6.5 Hz, 2.5 Hz, 1H), 7.25-7.19 (m, 2H), 7.11-6.99 (m, 2H), 6.95 (d, J=8.9 Hz, 2H), 6.31 (s, 1H), 5.04 (s, 2H), 3.84 (s, 3H), 3.70 (s, 3H), 3.06 (d, J=8.4 Hz, 1H), 2.05 (dd, J=12.8 Hz, 6.3 Hz, 4H), 1.90-1.84 (m, 2H), 1.70 (dd, J=7.1 Hz, 4.5 Hz, 2H).

### Example 32. 3-(3-Chloro-4-fluorophenyl)-1-((4-cyclopentyl-5-methyl-4H-1,2,4-triazol-3-yl)methyl)-1-(4-methoxyphenyl)urea

### Step 32-1: N-cyclopentylacetamide

¹H NMR (300 MHz, CDCl₃) δ 5.83 (s, 1H), 4.17 (dd, J=14.1 Hz, 7.0 Hz, 1H), 1.94 (s, 3H), 1.73-1.52 (m, 4H), 1.43-1.29 (m, 2H).

### Step 32-2: (E)-methyl N-cyclopentylacetimidate

¹H NMR (300 MHz, CDCl₃) δ 4.31-4.19 (m, 6H), 4.19 (dd, J=14.1 Hz, 7.0 Hz, 4H), 1.96 (s, 12H), 1.62 (tdd, J=8.5 Hz, 6.9 Hz, 2.1 Hz, 17H), 1.42-1.25 (m, 8H).

### Step 32-3: N-((4-cyclopentyl-5-methyl-4H-1,2,4-triazol-3-yl)methyl)-4-methoxyaniline

¹H NMR (300 MHz, CDCl₃) δ 6.88-6.81 (m, 2H), 6.77-6.66 (m, 2H), 4.72-4.53 (m, 1H), 4.40 (s, 2H), 3.78 (d, J=2.1 Hz, 3H), 2.54 (s, 2H), 1.99-1.65 (m, 7H).

### Step 32-4: 3-(3-Chloro-4-fluorophenyl)-1-((4-cyclopentyl-5-methyl-4H-1,2,4-triazol-3-yl)methyl)-1-(4-methoxyphenyl)urea

LC/MS [M+H]⁺: 460.15

¹H NMR (300 MHz, CDCl₃) δ 7.41 (dd, J=6.5 Hz, 2.5 Hz, 1H), 7.16 (d, J=8.9 Hz, 2H), 7.09-6.98 (m, 2H), 6.93 (d, J=8.9 Hz, 2H), 6.29 (s, 1H), 5.06 (s, 2H), 5.01-4.92 (m, 1H), 3.82 (s, 3H), 2.59 (s, 3H), 2.16 (s, 2H), 1.92 (d, J=3.5 Hz, 4H), 1.76 (d, J=3.0 Hz, 2H).

### Example 33. 1-(Benzo[d][1,3]dioxol-5-yl)-3-(3-chloro-4-fluorophenyl)-1-(1-(5,6,8,9-tetrahydro-[1,2,4]triazolo[4,3-d][1,4]oxazepin-3-yl)ethyl)urea

### Step 33-1: 1,4-Oxazepan-5-one

¹H NMR (300 MHz, CDCl₃) δ 6.73 (s, 1H), 3.94-3.69 (m, 4H), 3.35 (dd, J=8.3 Hz, 5.4 Hz, 2H), 2.80-2.59 (m, 2H).

### Step 33-2: (E)-5-Methoxy-2,3,6,7-tetrahydro-1,4-oxazepine

Crude

### Step 33-3: N-(1-(5,6,8,9-tetrahydro-[1,2,4]triazolo[4,3-d][1,4]oxazepin-3-yl)ethyl)benzo[d][1,3]dioxol-5-amine

¹H NMR (300 MHz, CDCl₃) δ 6.65 (d, J=8.3 Hz, 1H), 6.31 (d, J=2.3 Hz, 1H), 6.12 (dd, J=8.3 Hz, 2.3 Hz, 1H), 5.87 (s, 2H), 4.71 (q, J=6.6 Hz, 1H), 4.34-4.19 (m, 2H), 3.92-3.83 (m, 2H), 3.87-3.77 (m, 2H), 3.36-3.21 (m, 2H), 1.68 (d, J=6.7 Hz, 3H).

### Step 33-4: 1-(Benzo[d][1,3]dioxol-5-yl)-3-(3-chloro-4-fluorophenyl)-1-(1-(5,6,8,9-tetrahydro-[1,2,4]triazolo[4,3-d][1,4]oxazepin-3-yl)ethyl)urea

LC/MS [M+H]⁺: 476.1

¹H NMR (300 MHz, DMSO) δ 7.73 (s, 1H), 7.68 (dd, J=6.9 Hz, 2.4 Hz, 1H), 7.42-7.38 (m, 1H), 7.26 (t, J=9.1 Hz, 1H), 6.87 (d, J=8.2 Hz, 1H), 6.53 (s, 1H), 6.40 (s, 1H), 6.07 (s, 2H), 6.05-5.93 (m, 1H), 4.31-4.20 (m, 2H), 4.00-3.52 (m, 4H), 3.16-3.09 (m, 2H), 1.41 (d, J=6.9 Hz, 3H).

### Example 34. 3-(3-Chloro-4-fluorophenyl)-1-(4-(dimethylamino)phenyl)-1-((6,7,8,9-tetrahydro-5H-[1,2,4]triazolo[4,3-a]azepin-3-yl)methyl)urea

LC/MS [M+H]⁺: 457.9

¹H NMR (300 MHz, CDCl₃) δ 7.44 (dd, J=6.5 Hz, 2.5 Hz, 1H), 7.16-7.04 (m, 1H), 6.98 (dd, J=16.1 Hz, 8.7 Hz, 3H), 6.67 (d, J=8.9 Hz, 2H), 6.35 (s, 1H), 5.05 (s, 2H), 4.23 (d, J=4.5 Hz, 2H), 3.00 (d, J=12.1 Hz, 8H), 2.00-1.84 (m, 2H), 1.83-1.59 (m, 4H).

### Example 35. 3-(3-Chloro-4-fluorophenyl)-1-(1-methyl-1H-pyrazol-3-yl)-1-((6,7,8,9-tetrahydro-5H-[1,2,4]triazolo[4,3-a]azepin-3-yl)methyl)urea

### Step 35-1: Methyl 2-(1-methyl-1H-pyrazol-3-ylamino)acetate

LC/MS [M+H]⁺: 171

¹H NMR (300 MHz, Chloroform-d) δ 7.10 (d, J=2.2 Hz, 1H), 5.54 (d, J=2.3 Hz, 1H), 4.18 (d, J=15.0 Hz, 1H), 3.98 (s, 2H), 3.76 (s, 3H), 3.71 (s, 3H).

### Step 35-2: 2-(1-Methyl-1H-pyrazol-3-ylamino)acetohydrazide

¹H NMR (300 MHz, Chloroform-d) δ 8.01 (s, 1H), 7.13 (d, J=2.2 Hz, 1H), 5.53 (d, J=2.3 Hz, 1H), 3.89 (s, 2H), 3.73 (s, 3H).

### Step 35-3: 1-Methyl-N-((6,7,8,9-tetrahydro-5H-[1,2,4]triazolo[4,3-a]azepin-3-yl)methyl)-1H-pyrazol-3-amine

¹H NMR (300 MHz, Chloroform-d) δ 7.13 (d, J=2.2 Hz, 1H), 5.59 (s, 1H), 4.50 (s, 2H), 4.15-3.99 (m, 2H), 3.97-3.83 (m, 2H), 3.75 (s, 3H), 3.06-2.91 (m, 4H), 1.99-1.84 (m, 2H).

### Step 35-4: 3-(3-Chloro-4-fluorophenyl)-1-(1-methyl-1H-pyrazol-3-yl)-1-((6,7,8,9-tetrahydro-5H-[1,2,4]triazolo[4,3-a]azepin-3-yl)methyl)urea

LC/MS [M+H]⁺: 418

¹H NMR (300 MHz, Chloroform-d) δ 10.80 (s, 1H), 7.68 (dd, J=6.5 Hz, 2.6 Hz, 1H), 7.44-7.30 (m, 2H), 7.10 (t, J=8.8 Hz, 1H), 6.58 (d, J=2.4 Hz, 1H), 5.29 (s, 2H), 4.19 (m, 2H), 3.86 (s, 3H), 2.97 (m, 2H), 1.84 (m, 2H), 1.68 (m, 4H).

### Example 36. 3-(3-Chloro-4-fluorophenyl)-1-(isoxazol-3-yl)-1-((6,7,8,9-tetrahydro-5H-[1,2,4]triazolo[4,3-a]azepin-3-yl)methyl)urea

### Step 36-1: Methyl 2-(isoxazol-3-ylamino)acetate

¹H NMR (300 MHz, Chloroform-d) δ 8.09 (d, J=1.7 Hz, 1H), 5.92 (d, J=1.8 Hz, 1H), 4.51 (s, 1H), 4.07 (d, J=5.6 Hz, 2H), 3.82 (s, 3H).

### Step 36-2: 2-(Isoxazol-3-ylamino)acetohydrazide

¹H NMR (300 MHz, Methanol-d₄) δ 8.23 (d, J=1.8 Hz, 1H), 6.02 (d, J=1.8 Hz, 1H), 3.85 (s, 2H).

### Step 36-3: N-((6,7,8,9-tetrahydro-5H-[1,2,4]triazolo[4,3-a]azepin-3-yl)methyl)isoxazol-3-amine

LC/MS [M+H] 234

¹H NMR (300 MHz, Methanol-d₄) δ 8.24 (d, J=1.7 Hz, 1H), 6.00 (d, J=1.8 Hz, 1H), 4.52 (s, 2H), 4.15 (d, J=9.7 Hz, 3H), 2.99 (d, J=11.1 Hz, 3H), 1.94 (s, 2H), 1.73 (s, 5H).

### Step 36-4: 3-(3-Chloro-4-fluorophenyl)-1-(isoxazol-3-yl)-1-((6,7,8,9-tetrahydro-5H-[1,2,4]triazolo[4,3-a]azepin-3-yl)methyl)urea

LC/MS [M+H]⁺: 405

¹H NMR (300 MHz, Chloroform-d) δ 10.58 (s, 1H), 8.30 (d, J=1.8 Hz, 1H), 7.75 (dd, J=6.5 Hz, 2.6 Hz, 1H), 7.47-7.33 (m, 1H), 7.21-7.03 (m, 2H), 5.28 (s, 2H), 4.37-4.22 (m, 2H), 3.09-2.94 (m, 2H), 1.95-1.85 (m, 2H), 1.84-1.65 (m, 4H).

### Example 37. 3-(3-Chloro-4-fluorophenyl)-1-((4-cyclopropyl-5-methyl-4H-1,2,4-triazol-3-yl)methyl)-1-(4-methoxyphenyl)urea

### Step 37-1: N-((4-cyclopropyl-5-methyl-4H-1,2,4-triazol-3-yl)methyl)-4-methoxyaniline

¹H NMR (500 MHz, CDCl₃) δ 6.81 (d, J=8.9 Hz, 7H), 6.72 (d, J=8.9 Hz, 8H), 4.43 (d, J=5.1 Hz, 9H), 3.76 (s, 11H), 3.09-3.00 (m, 5H), 2.50 (s, 11H), 1.17 (dd, J=11.5 Hz, 4.7 Hz, 9H), 1.07-0.96 (m, 9H).

### Step 37-2: 3-(3-Chloro-4-fluorophenyl)-1-((4-cyclopropyl-5-methyl-4H-1,2,4-triazol-3-yl)methyl)-1-(4-methoxyphenyl)urea

LC/MS [M+H]⁺: 432.07

¹H NMR (500 MHz, CDCl₃) δ 7.50-7.43 (m, 3H), 7.07 (s, 1H), 7.00 (dd, J=15.0 Hz, 8.7 Hz, 3H), 6.40 (s, 1H), 5.08 (s, 2H), 3.86 (s, 3H), 3.12 (dd, J=7.1 Hz, 3.3 Hz, 1H), 2.50 (s, 3H), 1.23 (d, J=6.5 Hz, 2H), 1.00 (s, 2H).

### Example 38. Benzyl 3-((3-(3-chloro-4-fluorophenyl)-1-(4-methoxyphenyl)ureido)methyl)-8,9-dihydro-5H-[1,2,4]triazolo[4,3-d][1,4]diazepine-7(6H)-carboxylate

### Step 38-1: Benzyl 3-((4-methoxyphenylamino)methyl)-8,9-dihydro-5H-[1,2,4]triazolo[4,3-d][1,4]diazepin-7(6H)-carboxylate

Crude

### Step 38-2: Benzyl 3-((3-(3-chloro-4-fluorophenyl)-1-(4-methoxyphenyl)ureido)methyl)-8,9-dihydro-5H-[1,2,4]triazolo[4,3-d][1,4]diazepine-7(6H)-carboxylate

¹H NMR (500 MHz, CDCl₃) δ 7.40 (m, 6H), 7.21 (d, J=8.7 Hz, 2H), 7.05 (h, 2H), 6.98 (d, J=8.8 Hz, 2H), 6.23 (br, 1H), 5.22 (s, 2H), 5.01 (m, 2H), 4.37 (m, 2H), 3.85 (m, 5H), 3.74 (m, 2H), 3.19 (m, 2H).

### Example 39. 3-(3-Chloro-4-fluorophenyl)-1-(oxazol-2-yl)-1-((6,7,8,9-tetrahydro-5H-[1,2,4]triazolo[4,3-a]azepin-3-yl)methyl)urea

### Step 39-1: tert-Butyl oxazol-2-ylcarbamate

LC/MS [M+H] 185

¹H NMR (300 MHz, Chloroform-d) δ 10.37 (s, 1H), 7.44 (s, 1H), 6.94 (s, 1H), 1.56 (s, 9H).

### Step 39-2: Methyl 2-(tert-butoxycarbonyl(oxazol-2-yl)amino)acetate

LC/MS [M+H] 257

¹H NMR (300 MHz, Chloroform-d) δ 7.48 (s, 1H), 7.00 (s, 1H), 4.56 (s, 2H), 3.80 (s, 3H), 1.54 (s, 9H).

### Step 39-3: Methyl 2-(oxazol-2-ylamino)acetate

LC/MS [M+H] 157

¹H NMR (300 MHz, Chloroform-d) δ 7.20 (s, 1H), 6.80 (s, 1H), 5.21 (s, 1H), 4.15 (d, J=2.8 Hz, 2H), 3.81 (s, 3H).

### Step 39-4: 2-(oxazol-2-ylamino)acetohydrazide

Crude

### Step 39-5: N-((6,7,8,9-tetrahydro-5H-[1,2,4]triazolo[4,3-a]azepin-3-yl)methyl)oxazol-2-amine

LC/MS [M+H] 157

¹H NMR (300 MHz, Chloroform-d) δ 7.17 (s, 1H), 6.77 (s, 1H), 4.85 (s, 2H), 2.24 (d, J=29.1 Hz, 1H).

### Step 39-6: 3-(3-Chloro-4-fluorophenyl)-1-(oxazol-2-yl)-1-((6,7,8,9-tetrahydro-5H-[1,2,4]triazolo[4,3-a]azepin-3-yl)methyl)urea

LC/MS [M+H] 405

¹H NMR (300 MHz, Chloroform-d) δ 11.77 (s, 1H), 7.73 (dd, J=6.4 Hz, 2.4 Hz, 1H), 7.54 (s, 1H), 7.41-7.32 (m, 1H), 7.12 (t, J=8.8 Hz, 1H), 7.02 (s, 1H), 5.36 (s, 2H), 4.28-4.10 (m, 2H), 3.08-2.88 (m, 2H), 1.95-1.70 (m, 6H).

### Example 40. 3-(3-Chloro-4-fluorophenyl)-1-(6-methoxypyridin-3-yl)-1-((5,6,8,9-tetrahydro-[1,2,4]triazolo[4,3-d][1,4]oxazepin-3-yl)methyl)urea

### Step 40-1: Methyl 2-(6-methoxypyridin-3-ylamino)acetate

Crude

### Step 40-2: 2-(6-Methoxypyridin-3-ylamino)acetohydrazide

¹H NMR (300 MHz, CDCl₃) δ 7.54 (d, J=2.9 Hz, 1H), 6.96 (dd, J=8.8 Hz, 3.0 Hz, 1H), 6.62 (d, J=8.8 Hz, 1H), 3.83 (s, 3H), 3.78 (d, J 32 5.3 Hz, 2H).

### Step 40-3: 6-Methoxy-N-((5,6,8,9-tetrahydro-[1,2,4]triazolo[4,3-d][1,4]oxazepin-3-yl)methyl)pyridin-3-amine

Crude

### Step 40-4: 3-(3-Chloro-4-fluorophenyl)-1-(6-methoxypyridin-3-yl)-1-((5,6,8,9-tetrahydro-[1,2,4]triazolo[4,3-d][1,4]oxazepin-3-yl)methyl)urea

LC/MS [M+H]⁺: 449.14

¹H NMR (300 MHz, CDCl₃) δ 8.05 (d, J=2.6 Hz, 1H), 7.59 (dd, J=8.8 Hz, 2.8 Hz, 1H), 7.46-7.41 (m, 1H), 7.04 (dd, J=7.3 Hz, 4.1 Hz, 2H), 6.86 (d, J=8.8 Hz, 1H), 4.97 (s, 2H), 4.45-4.37 (m, 2H), 3.97 (s, 3H), 3.95-3.85 (m, 4H), 3.28-3.22 (m, 2H).

### Example 41. 1-(Benzo[d][1,3]dioxol-5-yl)-3-(3-chlorophenyl)-1-((5,6,7,8-tetrahydro-[1,2,4]triazolo[4,3-a]pyridin-3-yl)methyl)urea

### Step 41-1: 6-Methoxy-2,3,4,5-tetrahydropyridine

¹H NMR (300 MHz, CDCl₃) δ 3.59 (s, 3H), 3.50-3.42 (m, 2H), 2.21-2.06 (m, 2H), 1.70 (dd, J=7.7 Hz, 4.4 Hz, 2H), 1.59-1.49 (m, 2H).

### Step 41-2: N-((5,6,7,8-tetrahydro-[1,2,4]triazolo[4,3-a]pyridin-3-yl)methyl)benzo[d][1,3]dioxol-5-amine

¹H NMR (300 MHz, CDCl₃) δ 6.70 (d, J=8.3 Hz, 1H), 6.38 (d, J=2.3 Hz, 1H), 6.24-6.17 (m, 1H), 5.89 (s, 2H), 4.38 (s, 2H), 3.98 (t, J=5.9 Hz, 2H), 3.00 (t, J=6.3 Hz, 2H), 2.08-1.89 (m, 4H).

### Step 41-3: 1-(Benzo[d][1,3]dioxol-5-yl)-3-(3-chlorophenyl)-1-((5,6,7,8-tetrahydro-[1,2,4]triazolo[4,3-a]pyridin-3-yl)methyl)urea

LC/MS [M+H]⁺: 428.15

¹H NMR (300 MHz, CDCl₃) δ 7.42 (t, J=1.9 Hz, 1H), 7.21-7.15 (m, 1H), 7.12 (dt, J=8.2 Hz, 1.5 Hz, 1H), 7.03-6.98 (m, 1H), 6.85 (d, J=8.1 Hz, 1H), 6.82-6.73 (m, 2H), 6.42 (s, 1H), 6.06 (s, 2H), 5.01 (s, 2H), 4.13 (t, J=6.0 Hz, 2H), 2.99 (t, J=6.4 Hz, 2H), 2.10-1.99 (m, 2H), 1.99-1.87 (m, 2H).

### Example 42. 1-(Benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea

### Step 42-1: 5-Methoxy-3,6-dihydro-2H-1,4-oxazine

¹H NMR (300 MHz, CDCl₃) δ 4.05 (s, 2H), 3.74-3.61 (m, 5H), 3.54 (t, J=4.6 Hz, 2H).

### Step 42-2: N-((6,8-dihydro-5H-[1,2,4]triazolo[3,4-c][1,4]oxazin-3-yl)methyl)benzo[d][1,3]dioxol-5-amine

LC/MS [M+H]⁺: 276.1

¹H NMR (300 MHz, CDCl₃) δ 6.68 (d, J=8.3 Hz, 1H), 6.35 (d, J=2.3 Hz, 1H), 6.18 (dd, J=8.3 Hz, 2.4 Hz, 1H), 5.88 (s, 2H), 4.96 (s, 2H), 4.44 (s, 2H), 4.32-3.81 (m, 4H).

### Step 42-3: 1-(Benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea

LC/MS [M+H]⁺: 448.1

¹H NMR (300 MHz, CDCl₃) δ 7.44 (dd, J=6.5 Hz, 2.4 Hz, 1H), 7.20-6.95 (m, 2H), 6.87-6.76 (m, 3H), 6.29 (s, 1H), 6.06 (s, 2H), 4.98 (s, 4H), 4.38-4.21 (m, 2H), 4.19-3.98 (m, 2H).

### Example 43. 3-(3-Chloro-4-fluorophenyl)-1-(4-methoxyphenyl)-1-((5,6,7,8-tetrahydro-[1,2,4]triazolo[4,3-a]pyridin-3-yl)methyl)urea

### Step 43-1: 4-Methoxy-N-((5,6,7,8-tetrahydro-[1,2,4]triazolo[4,3-a]pyridin-3-yl)methyl)aniline

Crude

### Step 43-2: 3-(3-Chloro-4-fluorophenyl)-1-(4-methoxyphenyl)-1-((5,6,7,8-tetrahydro-[1,2,4]triazolo[4,3-a]pyridin-3-yl)methyl)urea

LC/MS [M+H]⁺: 432.1

¹H NMR (300 MHz, CDCl₃) δ 7.46 (dd, J=6.5 Hz, 2.4 Hz, 1H), 7.24 (d, J=8.8 Hz, 2H), 7.12-6.90 (m, 4H), 6.30 (s, 1H), 5.02 (s, 2H), 4.12 (t, J=5.9 Hz, 2H), 3.85 (s, 3H), 2.99 (t, J=6.3 Hz, 2H), 2.13-1.87 (m, 4H).

### Example 44. 3-(3-Chloro-4-fluorophenyl)-1-(6-methoxypyridin-3-yl)-1-((6,7,8,9-tetrahydro-5H-[1,2,4]triazolo[4,3-a]azepin-3-yl)methyl)urea

### Step 44-1: 6-Methoxy-N-((6,7,8,9-tetrahydro-5H-[1,2,4]triazolo[4,3-a]azepin-3-yl)methyl)pyridin-3-amine

LC/MS [M+H]⁺: 275.11

### Step 44-2: 3-(3-Chloro-4-fluorophenyl)-1-(6-methoxypyridin-3-yl)-1-((6,7,8,9-tetrahydro-5H-[1,2,4]triazolo[4,3-a]azepin-3-yl)methyl)urea

LC/MS [M+H]⁺: 445.09

¹H NMR (300 MHz, Chloroform-d) δ 7.98 (m, 1H), 7.54-7.42 (m, 2H), 7.08 (m, 1H), 7.01 (m, 1H), 6.80 (m, 1H), 6.45 (s, 1H), 5.00 (s, 2H), 4.26-4.08 (m, 2H), 3.94 (s, 2H), 3.06-2.91 (m, 2H), 1.89 (m, 2H), 1.83-1.66 (m, 4H).

### Example 45. Methyl 2-(5-((1-(benzo[d][1,3]dioxol-5-yl)-3-(3-chloro-4-fluorophenyl)ureido)methyl)-4H-1,2,4-triazol-3-yl)acetate

### Step 45-1: (E)-ethyl 3-amino-3-ethoxyacrylate

Crude

### Step 45-2: Ethyl 3-(2-(2-(benzo[d][1,3]dioxol-5-ylamino)acetyl)hydrazinyl)-3-iminopropanoate

LC/MS [M+H]⁺ 324.3

### Step 45-3: Ethyl 2-(5-((benzo[d][1,3]dioxol-5-ylamino)methyl)-4H-1,2,4-triazol-3-yl)acetate

LC/MS [M+H]⁺: 306.1

¹H NMR (300 MHz, DMSO) δ 6.64 (d, J=8.3 Hz, 1H), 6.34 (d, J=2.0 Hz, 1H), 6.16-5.94 (m, 1H), 5.82 (s, 2H), 4.21 (s, 2H), 4.09 (q, J=7.1 Hz, 2H), 3.73 (s, 2H), 1.17 (t, J=7.1 Hz, 3H).

### Step 45-4: 2-(5-((Benzo[d][1,3]dioxol-5-ylamino)methyl)-4H-1,2,4-triazol-3-yl)acetic acid

Crude

### Step 45-5: Methyl 2-(5-((benzo[d][1,3]dioxol-5-ylamino)methyl)-4H-1,2,4-triazol-3-yl)acetate

Crude

### Step 45-6: Methyl 2-(5-((1-(benzo[d][1,3]dioxol-5-yl)-3-(3-chloro-4-fluorophenyl)ureido)methyl)-4H-1,2,4-triazol-3-yl)acetate

LC/MS [M+H]⁺: 463.2

¹H NMR (300 MHz, CDCl₃) δ 7.47 (dd, J=6.4 Hz, 2.6 Hz, 1H), 7.18-7.08 (m, 1H), 7.02 (t, J=8.7 Hz, 1H), 6.85 (d, J=7.9 Hz, 1H), 6.75 (m, 2H), 6.37 (s, 1H), 6.05 (s, 2H), 4.86 (s, 2H), 3.86 (s, 2H), 3.76 (s, 3H).

### Example 46. 3-(3-Chloro-4-fluorophenyl)-1-(2-hydroxypyridin-4-yl)-1-((5,6,8,9-tetrahydro-[1,24]triazolo[4,3-d][1,4]oxazepin-3-yl)methyl)urea

### Step 46-1: Methyl 2-(2-methoxypyridin-4-ylamino)acetate

Crude

### Step 46-2: 2-(2-Methoxypyridin-4-ylamino)acetohydrazide

Crude

### Step 46-3: 2-Methoxy-N-((5,6,8,9-tetrahydro-[1,2,4]triazolo[4,3-d][1,4]oxazepin-3-yl)methyl)pyridin-4-amine

Crude

### Step 46-4: 3-(3-Chloro-4-fluorophenyl)-1-(2-methoxypyridin-4-yl)-1-((5,6,8,9-tetrahydro-[1,2,4]triazolo[4,3-d][1,4]oxazepin-3-yl) methyl)

Crude

### Step 46-5: 3-(3-Chloro-4-fluorophenyl)-1-(2-hydroxypyridin-4-yl)-1-((5,6,8,9-tetrahydro-[1,2,4]triazolo[4,3-d][1,4]oxazepin-3-yl) methyl)urea

LC/MS [M+H]⁺: 433.4

¹H NMR (300 MHz, DMSO-d₆) δ 9.09 (s, 1H), 9.06 (s, 1H), 7.82-7.70 (m, 1H), 7.60 (d, J=7.5 Hz, 1H), 7.41-7.26 (m, 2H), 6.56 (d, J=2.3 Hz, 1H), 6.36 (dd, J=7.5 Hz, 2.4 Hz, 1H), 5.13 (s, 2H), 4.34-4.20 (m, 2H), 3.87-3.66 (m, 4H), 3.14-3.02 (m, 2H).

### Example 47. 3-(3-Chloro-4-fluorophenyl)-1-(2-methoxypyridin-4-yl)-1-((5,6,8,9-tetrahydro-[1,2,4]triazolo[4,3-d][1,4]oxazepin-3-yl)methyl)urea

### Step 47-1: Ethyl 2-(tert-butoxycarbonylamino)acetate

¹H NMR (400 MHz, DMSO-d₆) δ 7.18 (t, J=6.2 Hz, 1H), 4.09 (q, J=7.2 Hz, 2H), 3.65 (d, J=6.2 Hz, 2H), 1.39 (s, 9H), 1.19 (t, J=7.1 Hz, 3H).

### Step 47-2: tert-Butyl 2-hydrazinyl-2-oxoethylcarbamate

¹H NMR (400 MHz, DMSO-d₆) δ 8.93 (s, 1H), 6.90 (t, J=6.2 Hz, 1H), 4.18 (s, 2H), 3.48 (d, J=6.1 Hz, 2H), 1.38 (s, 9H).

### Step 47-3: tert-Butyl (5,6,8,9-tetrahydro-[1,2,4]triazolo[4,3-d][1,4]oxazepin-3-yl)methylcarbamate

¹H NMR (400 MHz, DMSO-d₆) δ 7.42 (s, 1H), 4.25 (d, J=5.9 Hz, 2H), 4.21-4.07 (m, 2H), 3.84-3.68 (m, 4H), 3.15-2.97 (m, 2H), 1.38 (s, 9H).

### Step 47-4: 4-Chloro-2-methoxypyridine

¹H NMR (400 MHz, DMSO-d₆) δ 8.21 (d, J=5.8 Hz, 1H), 7.10 (d, J=2.3 Hz, 1H), 7.00 (dd, J=5.8 Hz, 2.3 Hz, 1H), 3.86 (s, 3H).

### Step 47-5: (5,6,8,9-tetrahydro-[1,2,4]triazolo[4,3-d][1,4]oxazepin-3-yl)methanamine hydrochloride

Crude

### Step 47-6: 2-Methoxy-N-((5,6,8,9-tetrahydro-[1,2,4]triazolo[4,3-d][1,4]oxazepin-3-yl)methyl)pyridin-4-amine

¹H NMR (400 MHz, Methanol-d₄) δ 7.82 (d, J=5.6 Hz, 1H), 7.25 (s, 1H), 6.30 (dd, J=6.1 Hz, 1.8 Hz, 1H), 6.16 (d, J=1.8 Hz, 1H), 4.67 (d, J=1.8 Hz, 2H), 4.32 (dt, J=6.0 Hz, 1.9 Hz, 2H), 3.88 (dd, J=4.1 Hz, 1.9 Hz, 4H), 3.81 (s, 3H), 3.25-3.14 (m, 2H).

### Step 47-7: 3-(3-Chloro-4-fluorophenyl)-1-(2-methoxypyridin-4-yl)-1-((5,6,8,9-tetrahydro-[1,2,4]triazolo[4,3-d][1,4]oxazepin-3-yl) methyl)urea

LC/MS [M+H]⁺: 447.1

¹H NMR (400 MHz, DMSO-d₆) δ 12.24 (s, 1H), 8.31 (d, J=5.9 Hz, 1H), 7.87 (dd, J=6.8 Hz, 2.7 Hz, 1H), 7.51 (ddd, J=9.0 Hz, 4.3 Hz, 2.7 Hz, 1H), 7.37 (t, J=9.1 Hz, 1H), 7.12 (d, J=2.2 Hz, 1H), 6.85 (dd, J=5.9 Hz, 2.1 Hz, 1H), 5.26 (s, 2H), 4.38-4.17 (m, 2H), 3.86 (s, 3H), 3.85-3.78 (m, 2H), 3.79-3.66 (m, 2H), 3.17-3.01 (m, 2H).

### Example 48. 3-(3-Chloro-4-fluorophenyl)-1-((5-(difluoromethyl)-4-methyl-4H-1,2,4-triazol-3-yl)methyl)-1-(6-methoxypyridin-3-yl)urea

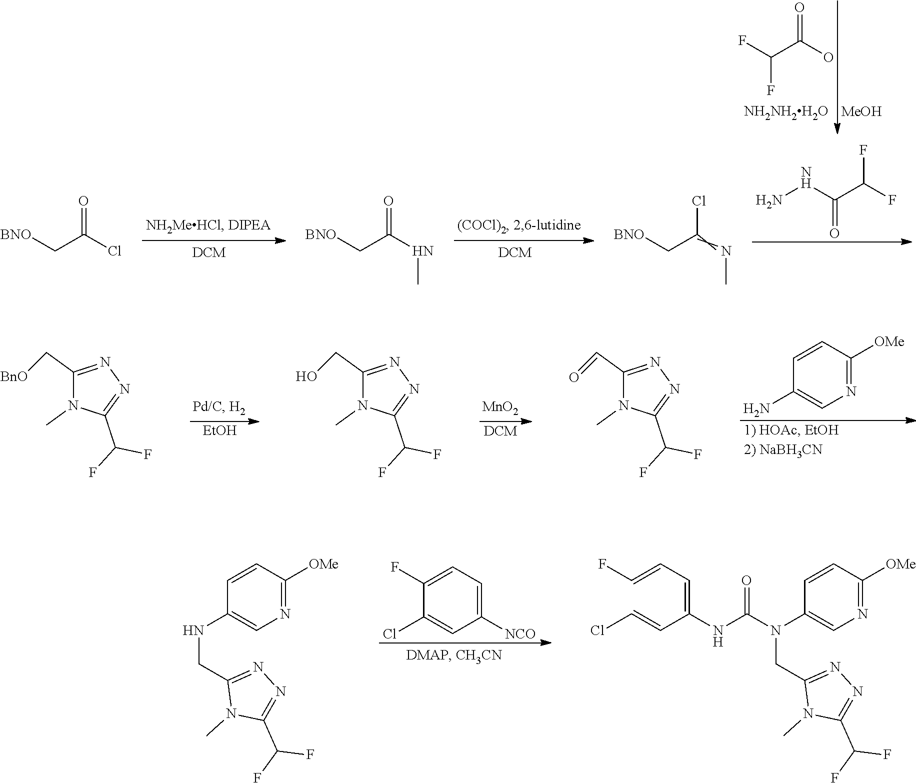

### Step 48-1: 2,2-Difluoroacetohydrazide

To a MeOH (200 mL) solution of 98% NH₂NH₂.H₂O (16.5 g, 323 mmol, 16 mL, 1.08 eq), methyl 2,2-difluoroacetate (33 g, 299.84 mmol, 1 eq) in MeOH (100 mL) was added dropwise over 1 hour at 0° C. The mixture was stirred at 15° C. for 1 hour and then at 70° C. for 1 hour. The mixture was concentrated to obtain the title compound (35 g, crude) as a colorless oil.

### Step 48-2: 2-(Benzyloxy)-N-methylacetamide

DIPEA (7.00 g, 54.2 mmol, 9.43 mL, 2.5 eq) was added to a mixture of methanamine hydrochloride (2.93 g, 43.3 mmol, 2 eq) in DCM (30 mL). The mixture was stirred at 15° C. for 10 minutes. Thereafter, 2-benzyloxyacetyl chloride (4.0 g, 21.7 mmol, 3.36 mL, 1 eq) in DCM (20 mL) was added thereto dropwise at 0° C. After the mixture was stirred at 15° C. for 1 hour, water (40 mL) was poured thereinto to terminate the reaction, and extraction with DCM (40 mL×3) was performed. The organic extracts were collected, dried over Na₂SO₄, filtered, and concentrated in vacuo. The residue was purified using silica gel chromatography (petroleum ether:ethyl acetate=5:1 to 1:1) to obtain the title compound (2.54 g, 60.18% yield, 92% purity) as a colorless oil.

### Step 48-3: 3-(Benzyloxymethyl)-5-(difluoromethyl)-4-methyl-4H-1,2,4-triazole

Oxalyl dichloride (1.82 g, 14.3 mmol, 1.26 mL, 1.1 eq) was added dropwise to a mixture of 2-(benzyloxy)-N-methylacetamide (2.54 g, 13.0 mmol, 1 eq) obtained in step 48-2 above and 2,6-dimethylpyridine (2.79 g, 26.1 mmol, 3.04 mL, 2 eq) in DCM (100 mL) at 0° C. The mixture was stirred at 15° C. for 1 hour. Thereafter, 2,2-difluoroacetohydrazide (1.87 g, 16.9 mmol, 1.3 eq) obtained in step 48-1 above was added thereto, and the mixture was stirred at 15° C. for 36 hours. After the mixture was concentrated to dryness in vacuo, saturated aqueous NaHCO₃ solution (50 mL) was added thereto, and the mixture was stirred at 100° C. for 3 hours. The mixture was cooled to room temperature and subjected to extraction with EA (60 mL×3). The organic layers were collected, dried over Na₂SO₄, filtered, and concentrated in vacuo. The residue was purified using silica gel chromatography (petroleum ether:ethyl acetate=5:1 to 1:1) and reversed-phase MPLC (0.1% FA condition) to obtain the title compound (515 mg, 15.6% yield) as a yellow oil.

### Step 48-4: (5-(Difluoromethyl)-4-methyl-4H-1,2,4-triazol-3-yl)methanol

Pd/C (0.05 g, 10% purity) was added to an EtOH (20 mL) solution of 3-(benzyloxymethyl)-5-(difluoromethyl)-4-methyl-4H-1,2,4-triazole (500 mg, 1.97 mmol, 1 eq) obtained in step 48-3 above. The mixture was stirred in H₂ (15 psi) at 50° C. for 16 hours. The reaction mixture was filtered and concentrated to obtain the title compound (330 mg, crude) as a black oil.

### Step 48-5: 5-(Difluoromethyl)-4-methyl-4H-1,2,4-triazole-3-carbaldehyde

A mixed DCM (30 mL) solution of (5-(difluoromethyl)-4-methyl-4H-1,2,4-triazol-3-yl)methanol (240 mg, 1.47 mmol, 1 eq) obtained in step 48-4 above and MnO₂ (1.28 g, 14.7 mmol, 10 eq) was stirred at 40° C. for 4 hours. The reaction mixture was filtered, and the filtrate was concentrated to obtain the title compound (205 mg, crude) as a white solid.

### Step 48-6: N-((5-(Difluoromethyl)-4-methyl-4H-1,2,4-triazol-3-yl)methyl)-6-methoxypyridin-3-amine

An EtOH (1 mL) solution of 6-methoxypyridin-3-amine (85 mg, 683 μmol, 1.1 eq) and the 5-(difluoromethyl)-4-methyl-4H-1,2,4-triazole-3-carbaldehyde (100 mg, 621 umol, 1 eq) obtained in step 48-5 above was stirred at 80° C. for 16 hours. Thereafter, NaBH₃CN (78 mg, 1.24 mmol, 2 eq) was added thereto, and the mixture was stirred at 0° C. for 0.5 hours, the reaction was then terminated with water (20 mL), and extraction with EA (15 mL×3) was performed. The organic layers were collected, washed with salt water (15 mL), dried over anhydrous Na₂SO₄, and filtered, and the filtrate was concentrated in vacuo. The residue was purified by Prep-TLC (petroleum ether:ethyl acetate=1:1) to obtain the title compound (40 mg, 24% yield) as a yellow gum.

### Step 48-7: 3-(3-Chloro-4-fluorophenyl)-1-((5-(difluoromethyl)-4-methyl-4H-1,2,4-triazol-3-yl)methyl)-1-(6-methoxypyridin-3-yl)urea

To a CH₃CN (3 mL) solution of N-((5-(difluoromethyl)-4-methyl-4H-1,2,4-triazol-3-yl)methyl)-6-methoxypyridin-3-amine (40 mg, 149 μmol, 1 eq) obtained in step 48-6 above and DMAP (18 mg, 149 μmol, 1 eq), 2-chloro-1-fluoro-4-isocyanatobenzene (51 mg, 297 μmol, 2 eq) was added at 15° C. The reaction mixture was stirred at 60° C. for 0.5 hours and then concentrated. The residue was purified by Prep-HPLC (column: Shim-pack C18 150 mm×25 mm×10 μm; mobile phase: [water (0.225% FA)-ACN]; B %: 33% to 63%, 10 min), and freeze-dried to obtain the title compound (19.8 mg, 29% yield, 95.1% purity) as a yellow solid.

LC/MS [M+H]⁺: 441.1

¹H NMR (400 MHz, CDCl₃) δ 8.14 (d, J=2.5 Hz, 1H), 7.66 (dd, J=2.7 Hz, 8.8 Hz, 1H), 7.48-7.42 (m, 1H), 7.11-7.02 (m, 2H), 6.94-6.76 (m, 2H), 6.17 (s, 1H), 5.09-4.98 (m, 2H), 4.05-3.92 (m, 6H).

The following compounds of Examples 49 to 53 were synthesized by conducting reactions in a manner similar to that in Example 48 using appropriate reactants in consideration of the structures of the title compounds. In these Examples, reactions for protection and deprotection of functional groups and/or introduction of additional substituents were additionally conducted depending on whether or not reactive sub-stituents were included. Hereinafter, the compounds of Examples 49 to 53, the intermediates thereof, and the data for identifying these compounds are sequentially disclosed.

### Example 49. 3-(3-Chloro-4-fluorophenyl)-1-(6-methoxypyridin-3-yl)-1-((4-methyl-5-(trifluoromethyl)-4H-1,2,4-triazol-3-yl)methyl)urea

### Step 49-1: 2-(Benzyloxy)-N-methylacetamide

Crude

### Step 49-2: 3-(Benzyloxymethyl)-4-methyl-5-(trifluoromethyl)-4H-1,2,4-triazole

Crude

### Step 49-3: (4-Methyl-5-(trifluoromethyl)-4H-1,2,4-triazol-3-yl)methanol

Crude

### Step 49-4: 4-Methyl-5-(trifluoromethyl)-4H-1,2,4-triazol-3-carbaldehyde

Crude

### Step 49-5: 6-Methoxy-N-((4-methyl-5-(trifluoromethyl)-4H-1,2,4-triazol-3-yl)methyl)pyridin-3-amine

Crude

### Step 49-6: 3-(3-Chloro-4-fluorophenyl)-1-(6-methoxypyridin-3-yl)-1-((4-methyl-5-(trifluoromethyl)-4H-1,2,4-triazol-3-yl)methyl)urea

LC/MS [M+H]⁺: 459.2

¹H NMR (400 MHz, DMSO-d₆) δ 8.30 (s, 1H), 8.17 (d, J=2.6 Hz, 1H), 7.75 (dd, J=2.7 Hz, 8.8 Hz, 1H), 7.68 (dd, J=2.5 Hz, 6.9 Hz, 1H), 7.42 (ddd, J=2.8 Hz, 4.3 Hz, 9.1 Hz, 1H), 7.32-7.25 (m, 1H), 6.90 (d, J=8.9 Hz, 1H), 5.08 (s, 2H), 3.88 (s, 3H), 3.81 (s, 3H).

### Example 50. 3-(3-Chloro-4-fluorophenyl)-1-((4-(2-hydroxyethyl)-5-(trifluoromethyl)-4H-1,2,4-triazol-3-yl)methyl)-1-(6-methoxypyridin-3-yl)urea

### Step 50-1: 2-(Benzyloxy)-N-(2-(tert-butyldimethylsilyloxy)ethyl)acetamide

Crude

### Step 50-2: 3-(Benzyloxymethyl)-4-(2-(tert-butyldimethylsilyloxy)ethyl)-5-(trifluoromethyl)-4H-1,2,4-triazole

Crude

### Step 50-3: (4-(2-(tert-Butyldimethylsilyloxy)ethyl)-5-(trifluoromethyl)-4H-1,2,4-triazol-3-yl)methanol

Crude

### Step 50-4: 4-(2-(tert-Butyldimethylsilyloxy)ethyl)-5-(trifluoromethyl)-4H-1,2,4-triazol-3-carbaldehyde

Crude

### Step 50-5: N-((4-(2-(tert-butyldimethylsilyloxy)ethyl)-5-(trifluoromethyl)-4H-1,2,4-triazol-3-yl)methyl)-6-methoxypyridin-3-amine

Crude

### Step 50-6: 1-((4-(2-(tert-Butyldimethylsilyloxy)ethyl)-5-(trifluoromethyl)-4H-1,2,4-triazol-3-yl)methyl)-3-(3-chloro-4-fluorophenyl)-1-(6-methoxypyridin-3-yl)urea

Crude

### Step 50-7: 3-(3-Chloro-4-fluorophenyl)-1-((4-(2-hydroxyethyl)-5-(trifluoromethyl)-4H-1,2,4-triazol-3-yl)methyl)-1-(6-methoxypyridin-3-yl)urea

LC/MS [M+H]⁺: 489.2

¹H NMR (400 MHz, CDCl₃-d) δ 8.24 (d, J=2.8 Hz, 1H), 7.80 (dd, J=2.8 Hz, 8.8 Hz, 1H), 7.40 (dd, J=2.4 Hz, 6.4 Hz, 1H), 7.08-7.02 (m, 1H), 7.02-6.96 (m, 1H), 6.86 (d, J=8.8 Hz, 1H), 6.23 (s, 1H), 5.08 (s, 2H), 4.45 (t, J=4.7 Hz, 2H), 3.97 (s, 3H), 3.97-3.94 (m, 2H).

### Example 51. 3-(3-Chloro-4-fluorophenyl)-1-((5-(difluoromethyl)-4-(2-methoxyethyl)-4H-1,2,4-triazol-3-yl)methyl)-1-(6-methoxypyridin-3-yl)urea

### Step 51-1: 2-(Benzyloxy)-N-(2-methoxyethyl)acetamide

¹H NMR (400 MHz, CDCl₃) δ 7.42-7.30 (m, 5H), 7.02-6.89 (m, 1H), 4.60-4.54 (m, 2H), 4.02-3.98 (m, 2H), 3.53-3.43 (m, 4H), 3.40-3.33 (m, 3H).

### Step 51-2: 3-(Benzyloxymethyl)-5-(difluoromethyl)-4-(2-methoxyethyl)-4H-1,2,4-triazole

¹H NMR (400 MHz, CDCl₃) δ 7.28 (s, 5H), 7.13-6.80 (m, 1H), 4.93-4.82 (m, 2H), 4.64-4.52 (m, 2H), 4.45-4.36 (m, 2H), 3.69-3.62 (m, 2H), 3.30-3.24 (m, 3H).

### Step 51-3: (5-(Difluoromethyl)-4-(2-methoxyethyl)-4H-1,2,4-triazol-3-yl)methanol

¹H NMR (400 MHz, CDCl₃) δ 7.05-6.70 (m, 1H), 4.86-4.76 (m, 2H), 4.42-4.32 (m, 2H), 4.22-4.08 (m, 1H), 3.69-3.62 (m, 2H), 3.31-3.24 (m, 3H).

### Step 51-4: 5-(Difluoromethyl)-4-(2-methoxyethyl)-4H-1,2,4-triazol-3-carbaldehyde

Crude

### Step 51-5: N-((5-(difluoromethyl)-4-(2-methoxyethyl)-4H-1,2,4-triazol-3-yl)methyl)-6-methoxypyridin-3-amine

Crude

### Step 51-6: 3-(3-Chloro-4-fluorophenyl)-1-((5-(difluoromethyl)-4-(2-methoxyethyl)-4H-1,2,4-triazol-3-yl)methyl)-1-(6-methoxypyridin-3-yl)urea

LC/MS [M+H]⁺: 485.2

¹H NMR (400 MHz, Chloroform-d) δ 8.26-8.21 (m, 1H), 7.82-7.75 (m, 1H), 7.50-7.44 (m, 1H), 7.12-6.96 (m, 3H), 6.91-6.84 (m, 1H), 6.26-6.18 (m, 1H), 5.09-5.04 (m, 2H), 4.64-4.58 (m, 2H), 4.03-3.96 (m, 3H), 3.70-3.65 (m, 2H), 3.31-3.27 (m, 3H).

### Example 52. 3-(3-Chloro-4-fluorophenyl)-1-((4-(2-methoxyethyl)-5-(trifluoromethyl)-4H-1,2,4-triazol-3-yl)methyl)-1-(6-methoxypyridin-3-yl)urea

LC/MS [M+H]⁺: 503.2

¹H NMR (400 MHz, DMSO-d₆) δ 8.33-8.30 (m, 1H), 8.19-8.15 (m, 1H), 7.78-7.72 (m, 1H), 7.70-7.65 (m, 1H), 7.44-7.38 (m, 1H), 7.31-7.23 (m, 1H), 6.94-6.87 (m, 1H), 5.11-5.05 (m, 2H), 4.48-4.40 (m, 2H), 3.92-3.86 (m, 3H), 3.63-3.56 (m, 2H), 3.23-3.17 (m, 3H).

### Example 53. 3-(3-Chloro-4-fluorophenyl)-1-((5-(difluoromethyl)-4-(2-hydroxyethyl)-4H-1,2,4-triazol-3-yl)methyl)-1-(6-methoxypyridin-3-yl)urea

### Step 53-1: 2-(Benzyloxy)-N-(2-(tert-butyldimethylsilyloxy)ethyl)acetamide

Crude

### Step 53-2: 3-(Benzyloxymethyl2-(tert-butyldimethylsilyloxy)ethyl)-5-(difluoromethyl)-4H-1,2,4-triazole

Crude

### Step 53-3: (4-(2-(tert-Butyldimethylsilyloxy)ethyl)-5-(difluoromethyl)-4H-1,2,4-triazol-3-yl)methanol

Crude

### Step 53-4: 4-(2-(tert-Butyldimethylsilyloxy)ethyl)-5-(difluoromethyl)-4H-1,2,4-triazol-3-carbaldehyde

Crude

### Step 53-5: N-((4-(2-(tert-butyldimethylsilyloxy)ethyl)-5-(difluoromethyl)-4H-1,2,4-triazol-3-yl)methyl)-6-methoxypyridin-3-amine

Crude

### Step 53-6: 1-((4-(2-(tert-Butyldimethylsilyloxy)ethyl)-5-(difluoromethyl)-4H-1,2,4-triazol-3-yl)methyl)-3-(3-chloro-4-fluorophenyl)-1-(6-methoxypyridin-3-yl)urea

Crude

### Step 53-7: 3-(3-Chloro-4-fluorophenyl)-1-((5-(difluoromethyl)-4-(2-hydroxyethyl)-4H-1,2,4-triazol-3-yl)methyl)-1-(6-methoxypyridin-3-yl)urea

LC/MS [M+H]⁺: 471.1

¹H NMR (400 MHz, CDCl₃) δ 8.23 (d, J=2.4 Hz, 1H), 7.80 (dd, J=2.8 Hz, 8.8 Hz, 1H), 7.41 (dd, J=2.4 Hz, 6.4 Hz, 1H), 7.08-7.03 (m, 1H), 7.02-6.78 (m, 3H), 6.24 (s, 1H), 5.09 (s, 2H), 4.53-4.41 (m, 2H), 4.03-3.98 (m, 2H), 3.97 (s, 3H).

### Experimental Example 1: Culturing of Cell and Treatment with Compound

HepAD38 cells were cultured in DMEM medium (Welgene) supplemented with 200 unit/mL penicillin, 200 μg/mL streptomycin, and 10% FBS. During maintenance and passage, the cells were cultured together with 0.4 μg/mL tetracycline. Two days before the treatment with compounds for inducing HBV replication was started, the medium was replaced with a tetracycline-free complete growth medium. The HepAD38 cells were seeded in a 48-well culture plate at a density of 1×10⁵ cells/well and treated with DMSO (0.2%, control) or a test compound (final concentration ranging from 1.5 nM to 0.37 μM).

### Experimental Example 2: Real-Time PCR for Intracellular HBV DNA

HepAD38 cells were treated with a compound for 65 hours and then recovered, and intracellular HBV DNA was extracted therefrom according to the protocol in the DNeasy Blood & Tissue Kit (Qiagen). The primers and probes used to quantify HBV DNA were 5′-CTCGTGGTGGACTTCTCTC-3′, 5′-CTGCAGGATGAAGAGGAA-3′, and 5′-/56-FAM/TGT CCT GGT/ZEN/TAT CGC TGG ATG TGT CT/3IABKFQ/-3′. The HBV DNA was amplified by real-time PCR assay using LightCycler 480 (Roche) (J. Virol., 2018, 92(16):e00339-18). All of the processes were confirmed in duplicate.

### Experimental Example 3: Cell Viability Assay

HepAD38 cells were cultured for 2 days in Dulbecco's Modified Eagle's medium (Welgene, LM001-05) supplemented with 10% FBS without tetracycline. The cells were seeded in 48-well culture plates (1×10⁵ cells/well) and treated with each compound at five concentrations (0 μM (0.8% DMSO as a control), 33 μM, 50 μM, 66 μM, and 100 μM). After 65 hours of treatment, viability was measured using EZ-Cytox cell viability assay kit (Daeil Lab Service) according to the manufacturer's instructions. The absorbance was measured at 450 nm using a spectrophotometer (Spark, Tecan). The IC₅₀, protein inhibition rate, cytotoxicity, and CLogP values of these compounds are presented in Table 1 below. Here, NVR-3-778 (denoted as NVR) was used as a positive control.

TABLE 1

| | IC₅₀ (μM) | Protein | | Cytotoxicity | | |
|---|---|---|---|---|---|---|
| | (NVR: | % inhibition | EC₅₀ | % Livability | CC₅₀ | CLog |
| Example | 0.40) | rate (@30 μM) | (μM) | (@100 μM) | (μM) | P |
| 1 | 0.02 | 72 | 1.3 (NVR: 5.2) | 95.0 | >100 | |
| 2 | 0.04 | 81 | — | 100.3 | >100 | |
| 3 | 0.19 | 89 | 1.6 (NVR: 4.8) | 91.9 | >100 | |
| 4 | 0.25 | 84 | 6.8 | — | >100 | |
| 5 | >3.3 | 9 | — | — | — | |
| 6 | 1.57 | 77 | 1.2 (NVR: 4.1) | — | — | |
| 7 | 0.03 | 80 | 1 (NVR: 4.1) | 96.4 | >100 | |
| 8 | 0.27 | 83 | 1.8 (NVR: 4.1) | 91.6 | >100 | |
| 9 | 0.75 | 5 | | | | |
| 10 | >3.3 | 85 | | | | |
| 11 | >3.3 | 4 | | | | |
| 12 | 0.11 | 95 | 2.5 | 93.2 | >100 | |
| 13 | 0.23 | 89 | 5 | 106 | >100 | |
| 14 | >0.55 | 92 | 2.8 | — | — | |
| 15 | >3.3 | 55.3 | 29 | — | — | |
| 16 | 0.124 | 80.9 | 2.2 (NVR: 5.2) | 93.9 | >100 | |
| 17 | >3.3 | 92.6 | 13 | — | — | |
| 18 | >3.3 | 96.8 | 4.8 (NVR: 5.2) | — | — | |
| 19 | >3.3 | 94 | — | — | — | |
| 20 | 0.89 | 3 | — | — | — | |
| 21 | >3.3 | 4 | 11 | — | — | |
| 22 | 0.4 | 43 | 2.6 | 92.9 | >100 | |
| 23 | 0.32 | 47 | 3.1 | 95.1 | >100 | |
| 24 | >3.3 | −1 | | | >100 | |
| 25 | >3.3 | −2 | — | — | — | |
| 26 | 0.184 | 2 | 6.2 | 91.5 | >100 | |
| 27 | 0.032 | 65 | 2.3 | 91.0 | >100 | |
| 28 | 0.604 | 36 | 4 | — | — | |
| 29 | >3.3 | 1 | — | — | — | |
| 30 | 0.74 | 32 | 3.6 | — | — | |
| 31 | 3.48 | −4 | 16 | — | — | |
| 32 | 0.32 | 23 | 4.9 | — | — | |
| 33 | 0.36 | 26 | 4.3 | 88.3 | >100 | |
| 34 | >3.3 | −7 | N/A | — | — | |
| 35 | >3.3 | 63.1 | — | — | — | |
| 36 | 0.40 | 77.6 | 1.5 | 90.4 | >100 | |
| 37 | >3.3 | 84.1 | — | — | — | |
| 38 | >3.3 | −1 | N/A | — | — | |
| 39 | >3.3 | 100.1 | 5.7 (NVR: 4.9) | — | — | |
| 40 | 0.084 | 82.8 | 1.5 (NVR: 3.9) | 88.6/100.3 | >100 | |
| 41 | 0.23 | 59 | 2.9 | 97.1 | >100 | |
| 42 | 0.21 | 20 | 6.8 | 83.7 | >100 | |
| 43 | 0.79 | 96.2 | 3.3 | 103.0 | >100 | |
| 44 | 0.05 | 82 | 1.4 | 103.0 | >100 | |
| 45 | >3.3 | 6 | — | — | — | |
| 46 | >1.1 | | | 90.5 | >100 | 1.55 |
| 47 | 0.092 | | | 81.2 | >100 | 2.05 |
| 48 | >1.1 | | | 102.0 | >100 | |
| 49 | 0.438 | | | 55.9 | >100 | |
| 50 | >1.1 | | | 101.2 | >100 | |
| 51 | >1.1 | | | 84.2 | >100 | |
| 52 | >1.1 | | | 55.4 | >100 | |
| 53 | >1.1 | | | 99.1 | >100 | |

Based on the above description, it will be understood by those skilled in the art that the present disclosure may be implemented in a different specific form without changing the technical spirit or essential characteristics thereof. Therefore, it should be understood that the above embodiment is not limitative, but illustrative in all aspects. The scope of the disclosure is defined by the appended claims rather than by the description preceding them, and thus all changes and modifications that fall within metes and bounds of the claims or equivalents of such metes and bounds are therefore intended to be embraced by the claims.

1. A compound represented by the following Chemical Formula 1 or a pharmaceutically acceptable salt of the compound: 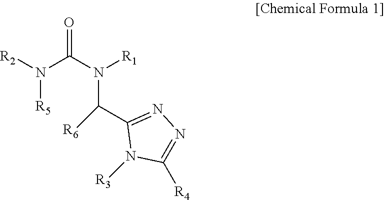
wherein:
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₅ and R₆ are each independently hydrogen or C₁₋₄ alkyl; and
a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₁₋₄ alkyl, C₁₋₄ alkylcarbonyl, C₁₋₄ alkoxycarbonyl, C₁₋₄ alkoxycarbonyl-C₁₋₄ alkyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl) amino, C₆₋₁₀ aryl, C₃₋₁₀ cycloalkyl, C₆₋₁₀ aryl-C₁₋₄ alkoxycarbonyl, and 5- to 10-membered heteroaryl.

2. The compound according to claim 1 or a pharmaceutically acceptable salt of the compound, wherein R₁ is phenyl, benzodioxolyl, dihydropyridinyl, cyclopentyl, indolyl, benzothiazolyl, pyrazolyl, isoxazolyl, oxazolyl, or pyridinyl.

3. The compound according to claim 1 or a pharmaceutically acceptable salt of the compound, wherein R₂ is phenyl, cyclopentyl, or pyridinyl.

4. The compound according to claim 1 or a pharmaceutically acceptable salt of the compound, wherein R₃ is hydrogen, methyl, phenyl, isopropyl, cyclopropyl, cyclopentyl, hydroxyethyl, or methoxyethyl.

5. The compound according to claim 1 or a pharmaceutically acceptable salt of the compound, wherein R₄ is methyl, isopropyl, phenyl, cyclopentyl, methoxycarbonylmethyl, difluoromethyl, or trifluoromethyl.

6. The compound according to claim 1 or a pharmaceutically acceptable salt of the compound, wherein R₃ and R₄ are connected to each other to form azepanyl, morpholinyl, oxazepanyl, diazepanyl, or piperidinyl including carbon and nitrogen to which R₃ and R₄ are bonded.

7. The compound according to claim 1 or a pharmaceutically acceptable salt of the compound, wherein R₅ is hydrogen or methyl.

8. The compound according to claim 1 or a pharmaceutically acceptable salt of the compound, wherein R₆ is hydrogen or methyl.

9. The compound according to claim 1 or a pharmaceutically acceptable salt of the compound, wherein a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, fluoro, chloro, methyl, isopropyl, methoxy, isopropoxy, methoxyethyl, tert-butoxycarbonyl, methoxycarbonylmethyl, hydroxyethyl, difluoromethyl, trifluoromethyl, methylamino, dimethylamino, cyclopropyl, cyclopentyl, benzyloxycarbonyl, and phenyl.

10. The compound according to claim 1 or a pharmaceutically acceptable salt of the compound, wherein the compound is:
1. 1-(benzo[d][1,3]dioxol-5-yl)-1-((6,7,8,9-tetrahydro-5H-[1,2,4]triazolo[4,3-a]azepin-3-yl)methyl)-3-m-tolylurea,
2. 1-(benzo[d][1,3]dioxol-5-yl)-3-(3-chloro-4-fluorophenyl)-1-((6,7,8,9-tetrahydro-5H-[1,2,4]triazolo[4,3-a]azepin-3-yl)methyl)urea,
3. 3-(3-chloro-4-fluorophenyl)-1-(4-methoxyphenyl)-1-((6,7,8,9-tetrahydro-5H-[1,2,4]triazolo[4,3-a]azepin-3-yl)methyl)urea,
4. 1-(benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea,
5. 1-(benzo[d][1,3]dioxol-5-yl)-3-(2-chlorophenyl)-1-((6,7,8,9-tetrahydro-5H-[1,2,4]triazolo[4,3-a]azepin-3-yl)methyl)urea,
6. 3-(3-chloro-4-fluorophenyl)-1-cyclopentyl-1-((6,7,8,9-tetrahydro-5H-[1,2,4]triazolo[4,3-a]azepin-3-yl)methyl)urea,
7. 1-(benzo[d][1,3]dioxol-5-yl)-3-(3-chloro-4-fluorophenyl)-1-((5,6,8,9-tetrahydro-[1,2,4]triazolo[4,3-d][1,4]oxazepin-3-yl)methyl)urea,
8. 1-(benzo[d][1,3]dioxol-5-yl)-3-(3-cyanophenyl)-1-((6,7,8,9-tetrahydro-5H-[1,2,4]triazolo[4,3-a]azepin-3-yl)methyl)urea,
9. 1-(3-chloro-4-fluorophenyl)-3-(4-methoxyphenyl)-1-methyl-3-((6,7,8,9-tetrahydro-5H-[1,2,4]triazolo[4,3-a]azepin-3-yl)methyl)urea,
10. 3-(3-chloro-4-fluorophenyl)-1-(1H-indol-6-yl)-1-((6,7,8,9-tetrahydro-5H-[1,2,4]triazolo[4,3-a]azepin-3-yl)methyl)urea,
11. 1-(benzo[d][1,3]dioxol-5-yl)-3-cyclopentyl-1-((6,7,8,9-tetrahydro-5H-[1,2,4]triazolo[4,3-a]azepin-3-yl)methyl)urea,
12. 3-(3-chloro-4-fluorophenyl)-1-(4-isopropoxyphenyl)-1-((6,7,8,9-tetrahydro-5H-[1,2,4]triazolo[4,3-a]azepin-3-yl)methyl)urea,
13. 3-(3-chloro-4-fluorophenyl)-1-((6,7,8,9-tetrahydro-5H-[1,2,4]triazolo[4,3-a]azepin-3-yl)methyl)-1-(4-(trifluoromethyl)phenyl)urea,
14. 3-(3-chloro-4-fluorophenyl)-1-(4-(methylamino)phenyl)-1-((6,7,8,9-tetrahydro-5H-[1,2,4]triazolo[4,3-a]azepin-3-yl)methyl)urea,
15. 3-(3-chloro-4-fluorophenyl)-1-((4,5-dimethyl-4H-1,2,4-triazol-3-yl)methyl)-1-(4-methoxyphenyl)urea,
16. 1-(benzo[d]thiazol-6-yl)-3-(3-chloro-4-fluorophenyl)-1-((6,7,8,9-tetrahydro-5H-[1,2,4]triazolo[4,3-a]azepin-3-yl)methyl)urea,
17. tert-butyl 5-(3-(3-chloro-4-fluorophenyl)-1-((6,7,8,9-tetrahydro-5H-[1,2,4]triazolo[4,3-a]azepin-3-yl)methyl)ureido)-1H-indole-1-carboxylate,
18. 3-(3-chloro-4-fluorophenyl)-1-(1H-indol-5-yl)-1-((6,7,8,9-tetrahydro-5H-[1,2,4]triazolo[4,3-a]azepin-3-yl)methyl)urea,
19. 3-(3-chloro-4-fluorophenyl)-1-(3-cyanophenyl)-1-((6,7,8,9-tetrahydro-5H-[1,2,4]triazolo[4,3-a]azepin-3-yl)methyl)urea,
20. 1-(benzo[d][1,3]dioxol-5-yl)-3-(6-methylpyridin-2-yl)-1-((6,7,8,9-tetrahydro-5H-[1,2,4]triazolo[4,3-a]azepin-3-yl)methyl)urea,
21. 3-(3-chloro-4-fluorophenyl)-1-((6,7,8,9-tetrahydro-5H-[1,2,4]triazolo[4,3-a]azepin-3-yl)methyl)-1-(3-(trifluoromethyl)phenyl)urea,
22. 3-(3-chloro-4-fluorophenyl)-1-(3,4-difluorophenyl)-1-((6,7,8,9-tetrahydro-5H-[1,2,4]triazolo[4,3-a]azepin-3-yl)methyl)urea,
23. 3-(3-chloro-4-fluorophenyl)-1-(4-methoxyphenyl)-1-(1-(6,7,8,9-tetrahydro-5H-[1,2,4]triazolo[4,3-a]azepin-3-yl)ethyl)urea,
24. 3-(3-chloro-4-fluorophenyl)-1-(4-methoxyphenyl)-1-((4-methyl-5-phenyl-4H-1,2,4-triazol-3-yl)methyl)urea,
25. 3-(3-chloro-4-fluorophenyl)-1-(4-methoxyphenyl)-1-((5-methyl-4-phenyl-4H-1,2,4-triazol-3-yl)methyl)urea,
26. 3-(3-chloro-4-fluorophenyl)-1-((4-isopropyl-5-methyl-4H-1,2,4-triazol-3-yl)methyl)-1-(4-methoxyphenyl)urea,
27. 3-(3-chloro-4-fluorophenyl)-1-(4-methoxyphenyl)-1-((5,6,8,9-tetrahydro-[1,2,4]triazolo[4,3-d][1,4]oxazepin-3-yl)methyl)urea,
28. 3-(3-chloro-4-fluorophenyl)-1-(4-cyanophenyl)-1-((6,7,8,9-tetrahydro-5H-[1,2,4]triazolo[4,3-a]azepin-3-yl)methyl)urea,
29. 3-(3-chloro-4-fluorophenyl)-1-((5-isopropyl-4-methyl-4H-1,2,4-triazol-3-yl)methyl)-1-(4-methoxyphenyl)urea,
30. 1-(benzo[d][1,3]dioxol-5-yl)-3-(3-chloro-4-fluorophenyl)-1-((5,6,7,8,9,10-hexahydro-[1,2,4]triazolo[4,3-a]azocin-3-yl)methyl)urea,
31. 3-(3-chloro-4-fluorophenyl)-1-((5-cyclopentyl-4-methyl-4H-1,2,4-triazol-3-yl)methyl)-1-(4-methoxyphenyl)urea,
32. 3-(3-chloro-4-fluorophenyl)-1-((4-cyclopentyl-5-methyl-4H-1,2,4-triazol-3-yl)methyl)-1-(4-methoxyphenyl)urea,
33. 1-(benzo[d][1,3]dioxol-5-yl)-3-(3-chloro-4-fluorophenyl)-1-(1-(5,6,8,9-tetrahydro-[1,2,4]triazolo[4,3-d][1,4]oxazepin-3-yl)ethyl)urea,
34. 3-(3-chloro-4-fluorophenyl)-1-(4-(dimethylamino)phenyl)-1-((6,7,8,9-tetrahydro-5H-[1,2,4]triazolo[4,3-a]azepin-3-yl)methyl)urea,
35. 3-(3-chloro-4-fluorophenyl)-1-(1-methyl-1H-pyrazol-3-yl)-1-((6,7,8,9-tetrahydro-5H-[1,2,4]triazolo[4,3-a]azepin-3-yl)methyl)urea,
36. 3-(3-chloro-4-fluorophenyl)-1-(isoxazol-3-yl)-1-((6,7,8,9-tetrahydro-5H-[1,2,4]triazolo[4,3-a]azepin-3-yl)methyl)urea,
37. 3-(3-chloro-4-fluorophenyl)-1-((4-cyclopropyl-5-methyl-4H-1,2,4-triazol-3-yl)methyl)-1-(4-methoxyphenyl)urea,
38. benzyl 3-((3-(3-chloro-4-fluorophenyl)-1-(4-methoxyphenyl)ureido)methyl)-8,9-dihydro-5H-[1,2,4]triazolo[4,3-d][1,4]diazepine-7(6H)-carboxylate,
39. 3-(3-chloro-4-fluorophenyl)-1-(oxazol-2-yl)-1-((6,7,8,9-tetrahydro-5H-[1,2,4]triazolo[4,3-a]azepin-3-yl)methyl)urea,
40. 3-(3-chloro-4-fluorophenyl)-1-(6-methoxypyridin-3-yl)-1-((5,6,8,9-tetrahydro-[1,2,4]triazolo[4,3-d][1,4]oxazepin-3-yl)methyl)urea,
41. 1-(benzo[d][1,3]dioxol-5-yl)-3-(3-chlorophenyl)-1-((5,6,7,8-tetrahydro-[1,2,4]triazolo[4,3-a]pyridin-3-yl)methyl)urea,
42. 1-(benzo[d][1,3]dioxol-5-yl)-3-(3-chloro-4-fluorophenyl)-1-((6,8-dihydro-5H-[1,2,4]triazolo[3,4-c][1,4]oxazin-3-yl)methyl)urea,
43. 3-(3-chloro-4-fluorophenyl)-1-(4-methoxyphenyl)-1-((5,6,7,8-tetrahydro-[1,2,4]triazolo[4,3-a]pyridin-3-yl)methyl)urea,
44. 3-(3-chloro-4-fluorophenyl)-1-(6-methoxypyridin-3-yl)-1-((6,7,8,9-tetrahydro-5H-[1,2,4]triazolo[4,3-a]azepin-3-yl)methyl)urea,
45. methyl 2-(5-((1-(benzo[d][1,3]dioxol-5-yl)-3-(3-chloro-4-fluorophenyl)ureido)methyl)-4H-1,2,4-triazol-3-yl)acetate,
46. 3-(3-chloro-4-fluorophenyl)-1-(2-hydroxypyridin-4-yl)-1-((5,6,8,9-tetrahydro-[1,2,4]triazolo[4,3-d][1,4]oxazepin-3-yl)methyl)urea,
47. 3-(3-chloro-4-fluorophenyl)-1-(2-methoxypyridin-4-yl)-1-((5,6,8,9-tetrahydro-[1,2,4]triazolo[4,3-d][1,4]oxazepin-3-yl)methyl)urea,
48. 3-(3-chloro-4-fluorophenyl)-1-((5-(difluoromethyl)-4-methyl-4H-1,2,4-triazol-3-yl)methyl)-1-(6-methoxypyridin-3-yl)urea,
49. 3-(3-chloro-4-fluorophenyl)-1-(6-methoxypyridin-3-yl)-1-((4-methyl-5-(trifluoromethyl)-4H-1,2,4-triazol-3-yl)methyl)urea,
50. 3-(3-chloro-4-fluorophenyl)-1-((4-(2-hydroxyethyl)-5-(trifluoromethyl)-4H-1,2,4-triazol-3-yl)methyl)-1-(6-methoxypyridin-3-yl)urea,
51. 3-(3-chloro-4-fluorophenyl)-1-((5-(difluoromethyl)-4-(2-methoxyethyl)-4H-1,2,4-triazol-3-yl)methyl)-1-(6-methoxypyridin-3-yl)urea,
52. 3-(3-chloro-4-fluorophenyl)-1-((4-(2-methoxyethyl)-5-(trifluoromethyl)-4H-1,2,4-triazol-3-yl)methyl)-1-(6-methoxypyridin-3-yl)urea, or
53. 3-(3-chloro-4-fluorophenyl)-1-((5-(difluoromethyl)-4-(2-hydroxyethyl)-4H-1,2,4-triazol-3-yl)methyl)-1-(6-methoxypyridin-3-yl)urea.

11. A method for preparing the compound according to claim 1 or a pharmaceutically acceptable salt of the compound, the method comprising reacting a compound represented by the following Chemical Formula 2 with a compound represented by the following Chemical Formula 3: 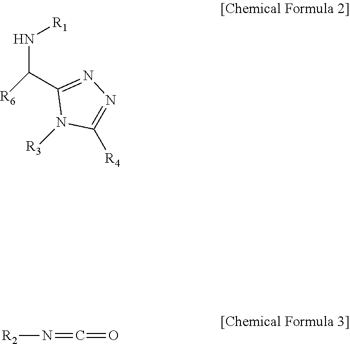
wherein:
R₁ is C₆₋₁₀ aryl, C₃₋₁₀ cycloalkyl, 5- to 10-membered heteroaryl, or 3- to 10-membered heterocyclyl;
R₂ is C₆₋₁₀ aryl, C₃₋₁₀ cycloalkyl, 5- to 10-membered heteroaryl, or 3- to 10-membered heterocyclyl;
R₃ and R₄ are each independently hydrogen, cyano, halogen, C₁₋₄ alkyl, C₆₋₁₀ aryl, C₃₋₁₀ cycloalkyl, C₁₋₄ alkoxycarbonyl-C₁₋₄ alkyl, C₁₋₄ alkenyl, C₁₋₄ haloalkyl, C₁₋₄ hydroxyalkyl, C₁₋₄ alkoxy, C₁₋₄ alkoxy-C₁₋₄ alkyl, C₁₋₄ alkoxy-C₁₋₄ alkenyl, 3- to 10-membered heterocyclyloxy-C₁₋₄ alkyl, or (4,4,5,5-tetra(C₁₋₄ alkyl)-1,3-dioxolanyl)-C₁₋₄ alkyl, or
R₃ and R₄ are connected to each other to form a 5- to 10-membered ring structure including nitrogen and carbon to which R₃ and R₄ are bonded;
R₆ is hydrogen or C₁₋₄ alkyl; and
a ring structure formed by connection of C₆₋₁₀ aryl, C₃₋₁₀ cycloalkyl, 5- to 10-membered heteroaryl, or 3- to 10-membered heterocyclyl and R₃ and R₄ to each other is unsubstituted or substituted with one or more selected from the group consisting of cyano, hydroxy, carboxyl, oxo, halogen, C₁₋₄ alkyl, C₁₋₄ alkylthio, C₁₋₄ alkoxy, C₁₋₄ alkoxy-C₋₁₋₄ alkyl, C₁₋₄ alkylcarbonyl, C₁₋₄ alkoxycarbonyl, C₁₋₄ alkoxycarbonyl-C₁₋₄ alkyl, C₁₋₄ alkylaminosulfonyl, C₁₋₄ alkylsulfonylamino, C₁₋₄ hydroxyalkyl, C₁₋₄ haloalkyl, C₁₋₄ alkylamino, di(C₁₋₄ alkyl)amino, C₆₋₁₀ aryl, C₃₋₁₀ cycloalkyl, C₆₋₁₀ aryl-C₁₋₄ alkoxycarbonyl, and 5- to 10-membered heteroaryl.

12. The preparation method according to claim 11, which is performed in an organic solvent in the presence of 4-dimethylaminopyridine (DMAP).

13. The preparation method according to claim 11, wherein the compound represented by Chemical Formula 2 is prepared by reacting
i) a compound represented by the following Chemical Formula 4 with a compound represented by Chemical Formula 5, or
ii) a compound represented by the following Chemical Formula 6 with a compound represented by Chemical Formula 7: 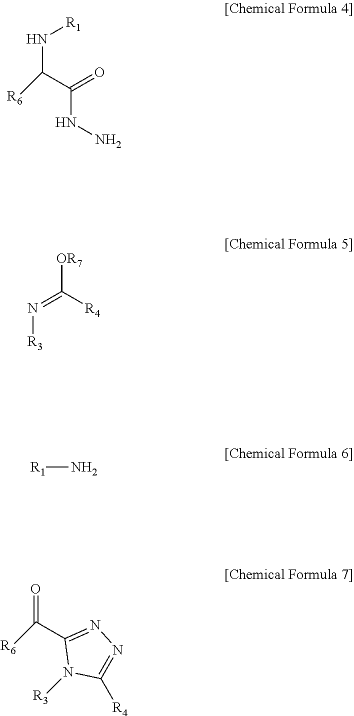
wherein:
R₇ is C₁₋₄ alkyl.

14. The preparation method according to claim 11, which further comprises conducting a reaction with R₅—X, wherein R₅ is hydrogen or C₁₋₄ alkyl, and X is halogen.

15. A composition for capsid assembly inhibition, comprising the compound according to claim 1 or a pharmaceutically acceptable salt of the compound.

16. An antiviral composition comprising the compound according to claim 1 or a pharmaceutically acceptable salt of the compound as an active ingredient.

17. A pharmaceutical composition for prevention or treatment of viral disease comprising the compound according to claim 1 or a pharmaceutically acceptable salt of the compound as an active ingredient.

18. The pharmaceutical composition according to claim 17, wherein the viral disease is an infectious disease caused by hepatitis B virus (HBV), hepatitis C virus (HCV), or human immunodeficiency virus (HIV).
